(19) United States
(12) Patent Application Publication
(10) Pub. No.: US 2022/0290091 A1
(43) Pub. Date: Sep. 15, 2022

(54) PROCESS CONTROL SYSTEMS FOR AUTOMATED CELL ENGINEERING SYSTEMS

(71) Applicants: LONZA WALKERSVILLE, INC., Walkersville, MD (US); OCTANE BIOTECH INC., Kingston (CA)

(72) Inventors: Eytan ABRAHAM, Walkersville, MD (US); PHIL DENSHAM, Walkersville, MD (US); Raelyn DANIELS, Walkersville, MD (US); NUALA TRAINOR, Walkersville, MD (US); IAN GRANT, Walkersville, MD (US); TIM SMITH, Walkersville, MD (US)

(21) Appl. No.: 17/635,638

(22) PCT Filed: Jul. 14, 2020
(86) PCT No.: PCT/US2020/041952

§ 371 Date: Feb. 15, 2022

Related U.S. Application Data
(60) Provisional application No. 62/874,119, filed on Jul. 15, 2019.

(51) Int. Cl. C12M 1/36; C12M 3/00; C12M 1/12; G16H 10/40

(52) U.S. Cl. CPC C12M 41/48; C12M 23/42; C12M 37/02; G16H 10/40; G06F 8/65

(57) ABSTRACT

Systems and methods for process control of automated cell engineering systems are provided. Automated cell engineering systems provide automated cell processing functionality. Automated process control systems provide control, interconnectivity, monitoring, data archival, software updating, and other oversight functions for automated cell engineering systems. Further, central control process systems provide control, monitoring, data archival, software updating, and other oversight functions for automated process control systems.

### RELATED MATTERS

This application claims the benefit of prior U.S. Provisional Patent Application Ser. No. 62/874,119, filed Jul. 15, 2019, which is hereby incorporated by reference in its entirety for all purposes.

### FIELD OF THE INVENTION

The present disclosure is related to control of automated cell engineering systems. In particular, the present disclosure relates to methods and systems providing process control and interconnectivity to automated cell engineering systems.

### BACKGROUND OF THE INVENTION

As anticipation builds about accelerated clinical adoption of advanced cell therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields, and particularly for T cell immunotherapies (see, e.g., Wang 2016).

Recent successful clinical results from immunotherapy trials using chimeric antigen receptor (CAR) T cells provide new hope to patients suffering from previously untreatable cancers (see, e.g., Lu 2017; Berdeja 2017; Kebriaei 2016). As these novel therapeutics move from the clinical trial stage to commercial scale-up, challenges arise related to cell manufacturing (see, e.g., Morrissey 2017).

The production of these cells may require significant manual involvement due to the patient-specific product. Automation of CAR T cell culture is particularly challenging due to the multiple sensitive unit operations, including cell activation, transduction, and expansion. Activation may be particularly important as the efficiency of this process can impact transduction and expansion.

Integration of cell activation, transduction and expansion into a commercial manufacturing platform is critical for the translation of these important immunotherapies to the broad patient population. For these life-saving treatments to be applicable to the global patient population, a shift in manufacturing techniques must be implemented to support personalized medicine. The benefits of automation have previously been described. These benefits include labor time savings associated with using automation as well as improved product consistency, decreased room classification, decreased clean room footprint, decreased training complexities, and improved scale-up and tracking logistics. Furthermore, software can be used to streamline the documentation processes by using automatically generated electronic batch records to provide a history of all processing equipment, reagents, patient identification, operator identification, in-process sensor data, and so forth.

Title 21 of the Code of Federal Regulations (Title 21 CFR Part 11) establishes US FDA regulations on electronic records. Specifically, part 11 defines the criteria under which electronic records are considered reliable, trustworthy, and equivalent to paper records. Part 11 defines rules for various record-keeping processes, including but not limited to validation, protection, access controls, personnel controls, reproduction, auditing, and others. One challenge of automated systems is maintaining compliance with Part 11.

The benefits of automation may not be fully realized without appropriate automated control. The present application provides technical solutions to technical problems related to automated control of automated cell engineering systems.

### SUMMARY OF THE INVENTION

In some embodiments provided herein is a method for controlling an automated cell engineering system configured to produce a cell culture. The method includes establishing, by a central computer system, a network connection with the automated cell engineering system; receiving, via the network connection, process information from the automated cell engineering system, the process information including one or more of temperature information, pH information, glucose concentration information, oxygen concentration information, component or patient identification information and optical density information; and providing a control signal, via the network connection, to cause the automated cell engineering system to adjust one or more process parameters of the automated cell engineering based on the received process information.

In another embodiment, a method for controlling a plurality of automated process control systems via a central control system is provided. The method includes establishing network connections with a plurality of computer systems corresponding to a plurality of automated process control systems, each configured to control a plurality of automated cell engineering systems configured for production of cell cultures; accessing, by the central control system, control information history of a first computer system from the plurality of computer systems; and providing to the first computer system at least one of a cell culture growth protocol update and a cell engineering software update.

In another embodiment, a method for automated production of a cell culture performed by an automated cell engineering system is provided. The method includes initiating a cell culture growth protocol within the automated cell engineering system; monitoring process information of the cell culture growth protocol; adjusting one or more parameters of the cell culture growth protocol based on the monitoring; arresting the cell culture growth protocol and recording a stage within the cell culture growth protocol at which the arresting occurred; and re-initiating the cell culture growth protocol at the stage within the cell culture growth protocol.

In another embodiment, a method for utilizing excess capacity within a network of automated cell engineering systems configured for automated production of cell cultures is provided. The method includes receiving, from a plurality of automated process control systems within the network, measures of excess capacity of the automated cell engineering systems; determining a capacity requirement according to patient requirements for a cell culture; matching the capacity requirement to a selected automated cell engineering system according to the measures of excess capacity; and transferring a biological sample to the selected cell engineering system for production of a cell culture.

In another embodiment, a method for automated production of a cell culture performed by an automated cell engineering system is performed. The method includes initiating a cell culture growth protocol within the automated cell engineering system; receiving, from an authorized user, an updated cell culture delivery requirement; and adjusting one or more parameters of the cell culture growth protocol based on the updated cell culture delivery requirement.

In another embodiment, a method for automated production of a cell culture performed by an automated cell engineering system is provided. The method includes initiating a cell culture growth protocol within the automated cell engineering system; monitoring one or more parameters of the cell culture growth protocol; projecting, according to the monitoring, a cell culture delivery date; and alerting an authorized user in advance of the cell culture delivery date.

### BRIEF DESCRIPTION OF THE FIGURES

FIG. 1 shows a generalized manufacturing process for a cell culture.

FIG. 2 shows a lab space containing exemplary cell engineering systems as described in embodiments herein.

FIG. 3 shows a cell culture production process that can be performed in a cell engineering system as described in embodiments herein.

FIGS. 4A-4C show an overview of an automated cell engineering system. FIG. 4A shows an automated cell engineering system in the closed configuration. FIG. 4B shows a Cassette that can be inserted into the automated cell engineering system. FIG. 4C shows an automated cell engineering system in the open configuration.

FIGS. 4D-4E show the location and orientation of a cell culture chamber utilized in an automated cell engineering system.

FIG. 4F shows a more detailed view of the cell culture chamber utilized in an automated cell engineering system.

FIG. 4G shows a process flow legend for an automated cell engineering system.

FIGS. 5A-5E show another configuration of an automated cell engineering system as described in embodiments herein. FIG. 5A shows a disposable cassette that can be loaded into the automated cell engineering system. FIG. 5B shows an automated cell engineering system in the open configuration. FIG. 5C shows the cassette loaded into the automated cell engineering system. FIG. 5D shows the automated cell engineering system in a closed configuration. FIG. 5E shows a detailed view of a cassette for use with the automated cell engineering system.

FIG. 5F shows the use of a syringe and a bag to sample from the cassette.

FIG. 6 shows the incorporation of an electroporation unit with a cell engineering system, in accordance with embodiments hereof.

FIG. 7 illustrates an automated process control system controlling an installation of automated cell tissue engineering system(s).

FIG. 8 illustrates an automated process control system consistent with embodiments hereof.

FIG. 9 illustrates a method of controlling an automated cell tissue engineering system.

FIG. 10 illustrates a central control process system controlling multiple automated process control system installations.

FIG. 11 illustrates a central control process system consistent with embodiments hereof.

FIG. 12 illustrates a method of controlling a plurality of automated process control systems.

FIG. 13 is a flow chart showing a process of controlling production of a cell culture.

FIG. 14 illustrates a capacity utilization service according to embodiments hereof.

FIG. 15 is a flow chart showing a process for utilizing excess capacity within a network of automated cell engineering systems configured for automated production of cell cultures.

FIG. 16 is a flow chart showing a process 1600 for automated production of a cell growth culture performed in an automated cell engineering system.

FIG. 17 is a flow chart showing a process for automated production of a cell growth culture performed in an automated cell engineering system.

### DETAILED DESCRIPTION OF THE INVENTION

The present disclosure provides systems and computer implemented methods of controlling and interacting with automated cell engineering systems. Automated cell engineering systems provide powerful tools for production of various engineered cells and tissues. Systems and methods described herein provide a technical solution to the technical problems involved with coordinating and controlling one or more automated cell engineering system. The systems and methods provided herein amplify the capabilities of automated cell engineering systems by facilitating control of, and access to, one or multiple automated cell engineering systems, whether they are collocated or non-collocated with each other and with control systems.

One automated cell engineering system consistent with embodiments hereof is the Cocoon™ platform, as described in greater detail below. The Cocoon™ platform is described in fuller detail in U.S. patent application Ser. No. 16/119,618, filed on Sep. 1, 2017, the contents of which are incorporated by reference herein in their entirety.

Automated Cell Processing

As described herein, installation and comprehensive validation of automated manufacturing provides a solution to logistical and operational challenges for production of engineered cells and tissues. An important approach to introducing automation to a production process is identifying the key modular steps where the operator applies a physical or chemical change to the production material, termed “unit operations.” In the case of cell manufacturing, this includes steps such as cell separation, genetic manipulation, proliferation, washing, concentration, and cell harvesting. Manufacturers often identify local process bottlenecks as the immediate opportunities for introducing automation. This is reflected in the technical operation spectrum of the majority of commercially available bioreactors, which tend to focus on discrete process steps. Process challenges in cell manufacturing (from sterility maintenance to sample tracking) are addressed herein by end-to-end automation that generates consistent cellular outputs while ameliorating inevitable process variability. The methods described herein also provide simplification, and the associated electronic records aid in complying with GMP standards (see, e.g., Trainor 2014).

Automation of Unit Operations and Key Process Sensitivities

The recent rapid progress of the clinical development of various cell cultures, including modified autologous T cells for cancer immunotherapy, has led to planning for the associated translation and scale up/out implications.

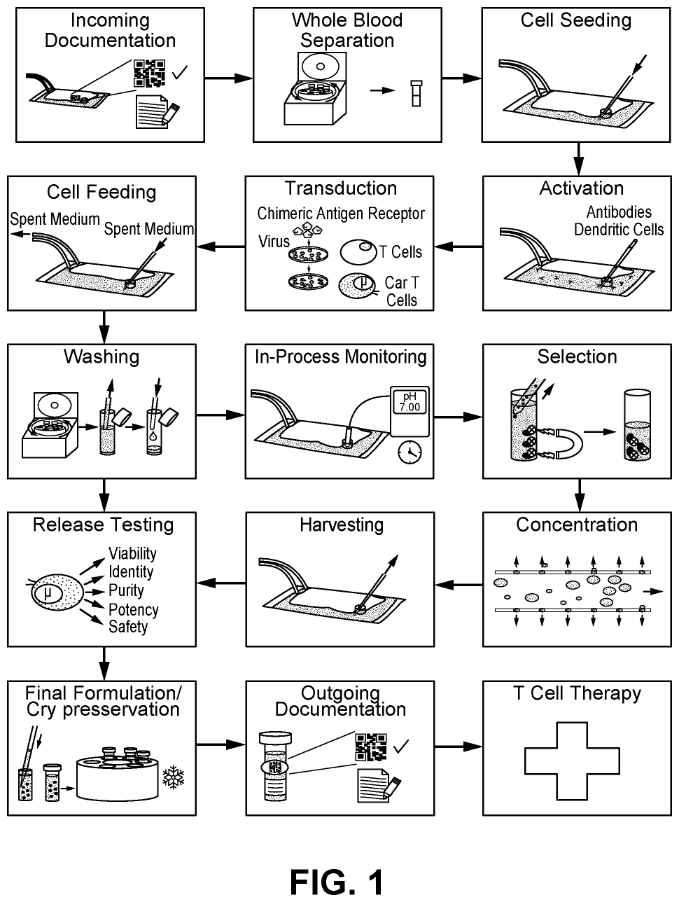

While specific cell culture growth protocols may vary for cell manufacturing, a generalized cell culture production process is illustrated in FIG. 1 (including production of autologous T cells). FIG. 1 describes unit operations of cell manufacturing, e.g., from initial processing of a patient blood sample to formulating output cells for autologous T cell therapy.

As described herein, to achieve cell manufacturing automation, the methods described herein provide for understanding the status of the cells at each transition point and how they are impacted by the specific unit operation. The micro-lot production for patient-specific therapies should be respectful of key process sensitivities that impact the feasibility of automation. Automation described herein successfully embraces various process steps.

Table 1 below highlights the challenges of some process steps identified for the automated production of cell cultures, including T cell automation. Note that for all unit operations, open transfer of cells between respective equipment is a key sensitivity due to the risk of contamination.

TABLE 1
Automation Challenges and Benefits

| Unit | Challenges of Key | |
|---|---|---|
| Operation | Process Steps | Benefit of Automating |
| Fractiona- | Highly variable based on | High purity of target starting |
| tion | donor cells and operator | population |
| | technique (see e.g., | More consistent and improved |
| | Nilsson 2008) | product |
| | Residual impurities can | |
| | impact performance | |
| Cell | Inhomogeneous cell | Homogenous automated |
| Seeding | distribution leads to | seeding strategy can improve |
| | variability in growth rates | consistency and potency |
| Activation | Stable contact between | Automated loading can ensure |
| | cells and activation | reproducibly homogeneous |
| | reagent | distribution and activation |
| | Uniform activation - | which can be difficult to |
| | homogeneous | consistently achieve with |
| | distribution | manual methods |
| Transduc- | Efficiency can be | Volume reduction prior to virus |
| tion | affected by the degree of | addition enables high degree of |
| | cell-virus mixing, which | cell-virus contact |
| | may vary based on | Time-based operation enables |
| | operator handling | cell transfer regardless of time |
| | Increased exposure time | of day |
| | may have negative | Closed system decreases risk |
| | impact on cells | to operator |
| Electro- | Efficiency can vary | Standardized protocols ensure |
| poration | based on operator | consistent results when |
| | mixing, washing and | upstream and downstream |
| | concentration technique | steps are integrated |
| Feeding | Timing of media | Biofeedback can optimize |
| | exchange needs to | feeding schedule (see, e.g., Lu |
| | consider nutritional | 2013) and minimize media use |
| | requirements based on | Components can be stored at |
| | cell growth (see, e.g., | refrigerated temperatures to |
| | Bohenkamp 2002), and | prolong stability and |
| | the component stability | automatically pre-warmed |
| | at 37° C. | before use |
| Selection | Extensive handling steps | Full automation improves |
| | can result in cell loss | consistency |
| | Operator variability | |
| Harvest | Acellular materials (such | Cells automatically transferred |
| | as cell separation beads) | from culture vessel regardless |
| | to be removed prior to | of time of day |
| | final formulation (see | Improved final yield |
| | e.g., Hollyman 2009) | consistency over manual |
| | Manual pipetting | pipetting |
| | variability can impact | |
| | final yield | |
| Washing | Aggressive washing may | Gentle washing, filtration, or |
| | induce shear stress or | sedimentation without moving |
| | cause cell loss during | the culture vessels, can be |
| | supernatant removal | utilized to reduce cell loss and |
| | | remove residuals |
| Concen- | Cell recovery may vary | Automated volume reduction |
| tration | by operator during | reduces operator variability |
| | aspiration | Filtration methods also |
| | | minimize cell loss |
| Formula- | Product must be well | Automated mixing ensures |
| tion | mixed | homogenous distribution of |
| | Small working volumes | cells in final formulation |
| | magnify impact of | Automated volume addition |
| | volume inaccuracies | removes risk of manual |
| | Viability decreases with | pipetting error or variability |
| | longer exposure times to | Increased automation reduces |
| | cryopreservative | variability in temperature |
| | | sensitive steps |

Tailoring the automation of a manual process around the sensitivities listed in Table 1 can support successful translation, maintenance, or improvement on the performance of the cell therapy.

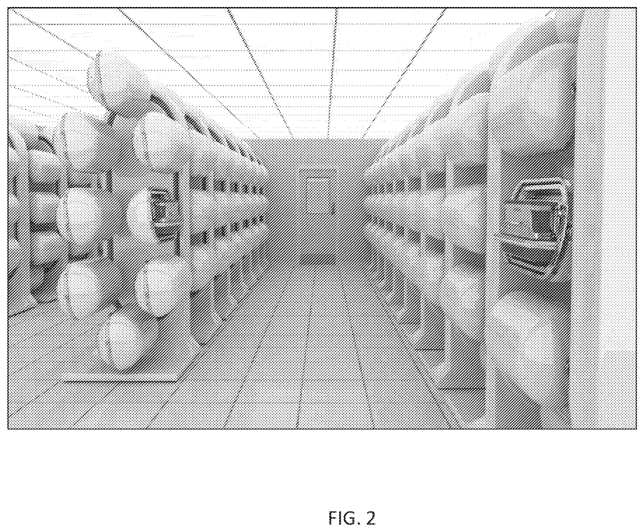

A single all-in-one system can offer significantly greater space efficiency to minimize the required footprint in expensive GMP clean rooms. For example, as shown in FIG. 2, fully integrated automated systems are designed to maximize required footprint to reduce expensive GMP clean room space. FIG. 2 shows e.g., 96 patient-specific end-to-end units running in a standard lab space.

A single system also provides greater ease of data tracking, whereas discrete systems may not offer compliant software that links together all electronic data files. Software platforms such as VINETI (Vineti Ltd) and TRAKCEL (TrakCel Ltd) allow electronic monitoring and organization of supply chain logistics. However, single all-in-one culture systems can go further still by incorporating a history of both processing events, process information, biomonitoring culture conditions (also referred to as production information), and user control history associated with each unit operation into a batch record. Accordingly, the benefits of end-to-end integration offer a significant competitive advantage.

Commercial Platforms for Integration of Unit Operations

Clinical trial success in a number of autologous cell therapies, especially immunotherapy for blood-based cancers, has highlighted the importance of enabling translation of new clinical protocols to robust production platforms to meet projected clinical demand (see, e.g., Levine 2017; Locke 2017). For autologous therapies, processing each patient-specific cell treatment suitably utilizes comprehensive manufacturing activities and operations management. The methods herein link unit operations in a turnkey automated system to achieve process optimization, security, and economy.

The challenge in designing an autologous process is two-fold. Firstly, unlike allogeneic manufacturing in which separate processing steps can occur in physically separate and optimized pieces of equipment, scaled-out autologous platforms suitably perform all of the necessary steps in a single closed, self-contained automated environment. Secondly, unlike an allogeneic process in which every run theoretically starts with a high-quality vial from a cell bank, with known quality and predictable process behavior, the starting material in an autologous process is highly variable, and generally comes from individuals with compromised health.

Thus, provided herein are methods that are able to sense culture conditions and respond accordingly as a sophisticated bioreactor, by controlling factors such as physical agitation, pH, feeding, and gas handling. Furthermore, there are significantly different challenges with technology transfer related to autologous treatments compared to allogeneic treatments. Autologous products may have greater restrictions on stability between the manufacturing process and the patient treatment. Sites can be located globally rather than at a single center. Having a locked down (e.g., fully enclosed) all-in-one system significantly improves the technology transfer process between sites.

While source variability cannot be eliminated, automation helps to remove variability of the final autologous product through standardization and reproducibility. This practice is adopted by leading cell system providers to obtain a cell performance reference point via biosensors that monitor the status of the active cell cultures. In end-to-end integration, output from any specific stage in the process should be within acceptable parameters for the onward progression of the process.

As described herein, in embodiments, the methods provided utilize the Cocoon™ platform (Octane Biotech (Kingston, ON)), which integrates multiple unit operations in a single turnkey platform (see e.g., U.S. Published Patent Application No. 2019/0169572, the disclosure of which is incorporated by reference herein in its entirety). It is understood, however, that other fully or partially automated cell culture apparatus may be used according to embodiments hereof, including those commercially available such as PRODIGY available from Miltenyi Biotech, Inc., XURI and SEFIA from General Electric Healthcare, and systems available from Atvio Biotech Ltd. Multiple cell culture growth protocols are provided with very specific cell processing objectives. To provide efficient and effective automation translation, the methods described utilize the concept of application-specific/sponsor-specific disposable cassettes that combine multiple unit operations—all focused on the core requirements of the final cell therapy product.

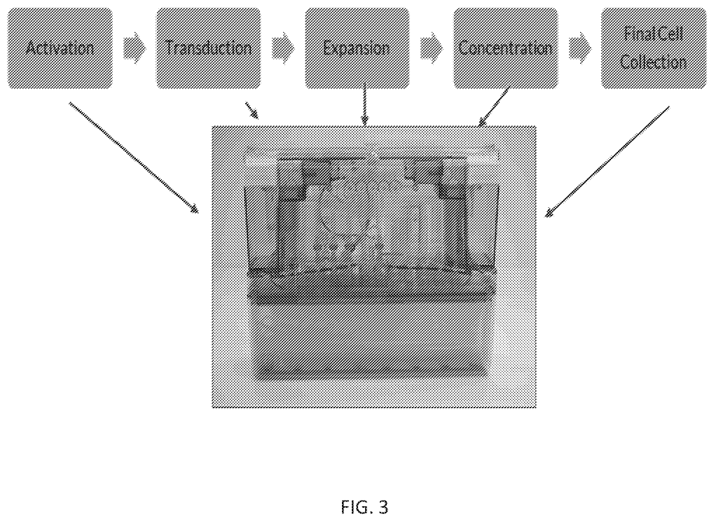

The methods described herein have been used to expand CAR-T cells (including activation, viral transduction and expansion, concentration and washing) in a fully-integrated closed automation system (FIG. 3).

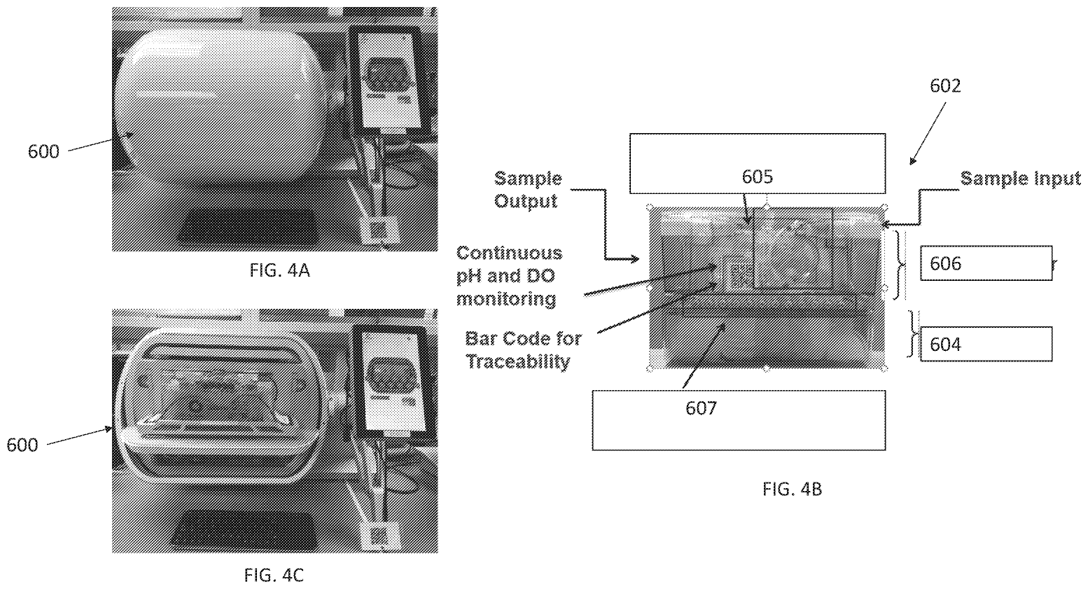

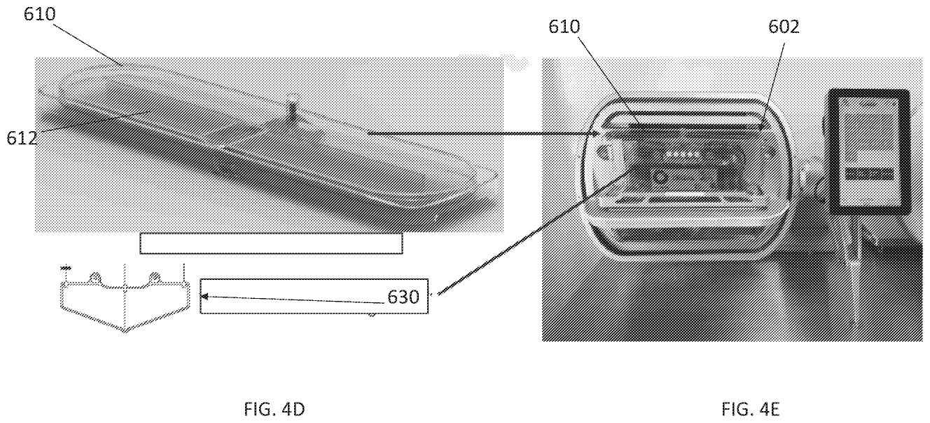

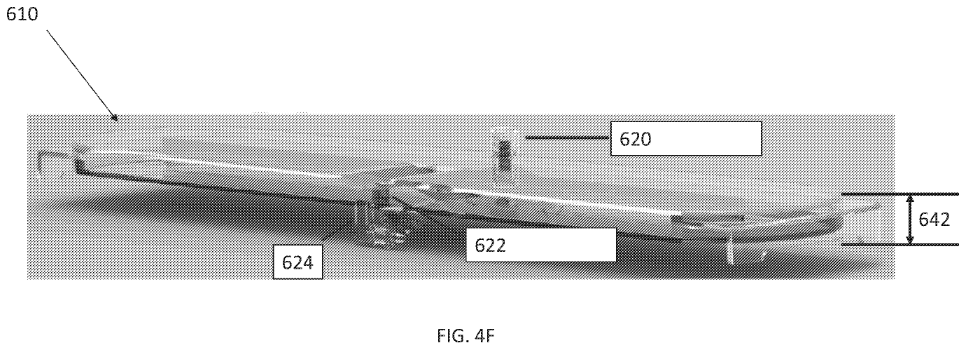

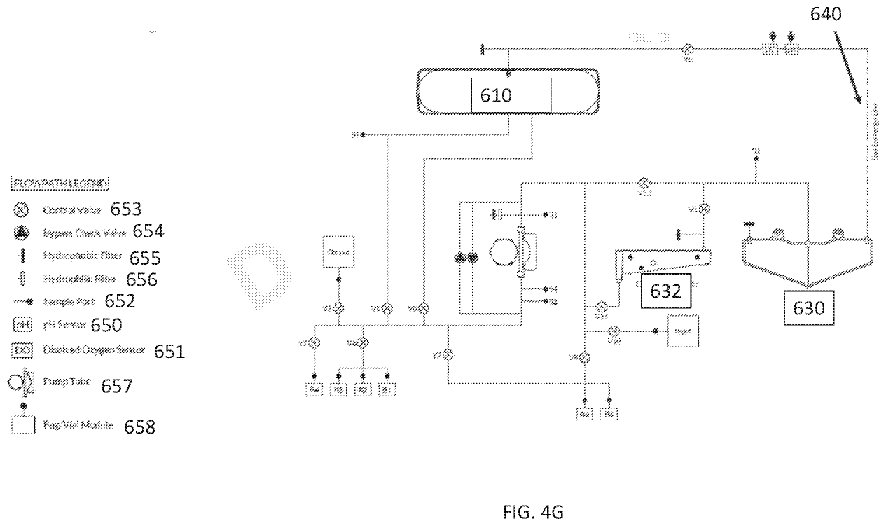

Automated Cell Engineering Systems. In some embodiments, the methods described herein are performed by a fully enclosed, automated cell engineering system 600 (see FIGS. 4A, 4B), suitably having instructions thereon for performing activating, transducing, expanding, concentrating, and harvesting steps, of cell cultures. Cell engineering systems (also called automated cell engineering systems throughout) provide for the automated production of cell cultures. As used herein “cell cultures” refers to any suitable cell type, including individual cells, as well as multiple cells or cells that may form into tissue structures. Exemplary cell cultures include blood cells, skin cells, muscle cells, bone cells, cells from various tissues and organs, etc., In embodiments, genetically modified immune cells, including CAR T cells, as described herein, can be produced. Exemplary automated cell engineering systems are also called Cocoon™, or Cocoon™ system throughout.

For example, a user can provide a cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like), the cell engineering system is able to carry out methods of producing an engineering cell culture, including genetically modified immune cell cultures, including CAR T cells, without further input from the user. At the end of the automated production process, the cell engineering system may alert the user (e.g., by playing an alert message or sending a mobile app alert) for collecting the produced cells. In some embodiments, the fully enclosed cell engineering system includes sterile cell culture chambers. In some embodiments, the fully enclosed cell engineering system minimizes contamination of the cell cultures by reducing exposure of the cell culture to non-sterile environments. In additional embodiments, the fully enclosed cell engineering system minimizes contamination of the cell cultures by reducing user handling of the cells.

As described herein, the cell engineering systems suitably include a cassette 602 (see FIG. 4B). As used herein a “cassette” refers to a largely self-contained, removable and replaceable element of a cell engineering system that includes one or more chambers for carrying out the various elements of the methods described herein, and suitably also includes one or more of a cell media, an activation reagent, a vector, etc. A cassette can include a flexible bag, rigid container, or other construction element. In some aspects, the cassette can be configured for a single-use.

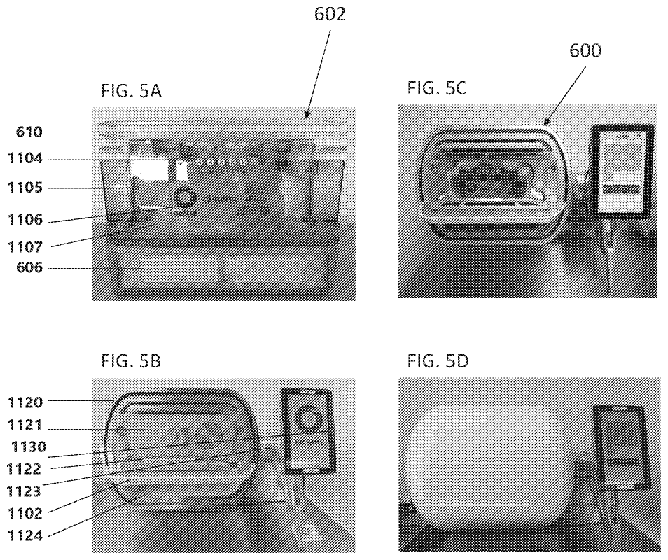

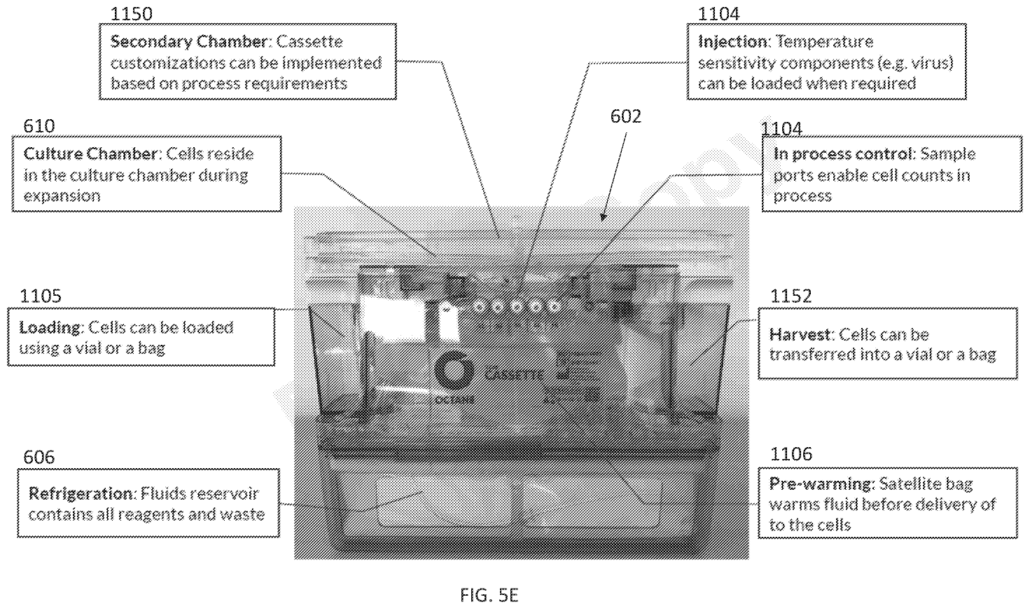

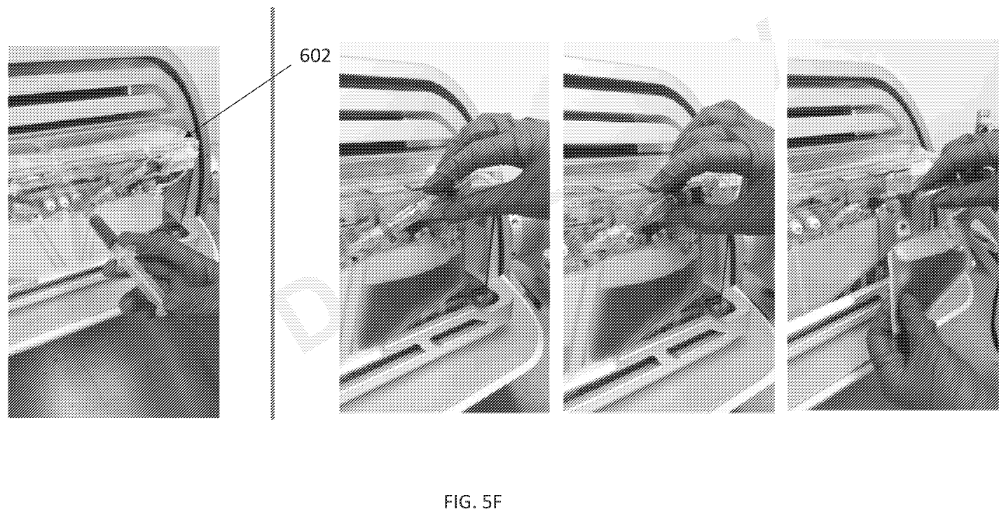

FIG. 4B shows an embodiments of a cassette 602 in accordance with embodiments hereof. In embodiments, cassette 602 includes a low temperature chamber 604, suitably for storage of a cell culture media, as well as a high temperature chamber 606, suitably for carrying out activation, transduction and/or expansion of an immune cell culture. Suitably, high temperature chamber 606 is separated from low temperature chamber 604 by a thermal barrier 1102 (see FIG. 5b). As used herein “low temperature chamber” refers to a chamber, suitably maintained below room temperature, and more suitably from about 4° C. to about 8° C., for maintenance of cell media, etc., at a refrigerated temperature. The low temperature chamber can include a bag or other holder for media, including about 1 L, about 2 L, about 3 L, about 4 L, or about 5 L of fluid. Additional media bags or other fluid sources can be connected externally to the cassette and connected to the cassette via an access port.

As used herein “high temperature chamber” refers to chamber, suitably maintained above room temperature, and more suitably maintained at a temperature to allow for cell proliferation and growth, i.e., between about 35-40° C., and more suitably about 37° C.

In embodiments, high temperature chamber 606 suitably includes a cell culture chamber 610 (also called proliferation chamber or cell proliferation chamber throughout), as shown in FIG. 4d and FIG. 4e.

The cassettes can, in some aspects, further include one or more fluidics pathways connected to the cell culture chamber, wherein the fluidics pathways provide recirculation, removal of waste and homogenous gas exchange and distribution of nutrients to the cell culture chamber without disturbing cells within the cell culture chamber. Cassette 602 also further includes one or more pumps 605, including peristaltic pumps, for driving fluid through the cassette, as described herein, as well as one or more valves 607, for controlling the flow through the various fluidic pathways.

In exemplary embodiments, as shown in FIG. 4d, cell culture chamber 610 is flat and non-flexible chamber (i.e., made of a substantially non-flexible material such as a plastic) that does not readily bend or flex. The use of a non-flexible chamber allows the cells to be maintained in a substantially undisturbed state. As shown in FIG. 4e, cell culture chamber 610 is oriented so as to allow the immune cell culture to spread across the bottom 612 of the cell culture chamber. As shown in FIG. 4e, cell culture chamber 610 is suitably maintained in a position that is parallel with the floor or table, maintaining the cell culture in an undisturbed state, allowing the cell culture to spread across a large area of the bottom 612 of the cell culture chamber. In embodiments, the overall thickness of cell culture chamber 610 (i.e., the chamber height 642) is low, on the order of about 0.5 cm to about 5 cm. Suitably, the cell culture chamber has a volume of between about 0.50 ml and about 300 ml, more suitably between about 50 ml and about 200 ml, or the cell culture chamber has a volume of about 180 ml. The use of a low chamber height 642 (less than 5 cm, suitably less than 4 cm, less than 3 cm, or less than 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss.

As described herein, in exemplary embodiments the cassette is pre-filled with one or more of a cell culture, a culture media, an activation reagent, and/or a vector, including any combination of these. In further embodiments, these various elements can be added later via suitable injection ports, etc.

As described herein, in embodiments, the cassettes suitably further include one or more of a pH sensor, a glucose sensor, an oxygen sensor, a carbon dioxide sensor, a lactic acid sensor/monitor, and/or an optical density sensor. The cassettes can also include one or more sampling ports and/or injection ports. Examples of such sampling ports and injection ports (1104) are illustrated in FIG. 5a. and can include an access port for connecting the cartridge to an external device, such as an electroporation unit or an additional media source. FIG. 5a also shows the location of the cell input 1105, reagent warming bag 1106 which can be used to warm cell media, etc., as well as the culture zone 1107, which holds various components for use in the culture media, including for example, cell media, vectors, nutrients and waste products, etc.

FIG. 5b shows an automated cell engineering system with cassette 602 removed. Visible in FIG. 5b are components of the cell engineering system, including gas control seal 1120, warming zone 1121, actuators 1122, pivot 1123 for rocking or tilting the cell engineering system as desired, and low temperature zone 1124 for holding low temperature chamber 604. Also shown is an exemplary user interface 1130, which can include a bar code reader and/or QR code reader, and the ability to receive using inputs by touch pad or other similar device. The user interface 1130 that may further include a component identification sensor such as a bar code reader, QR code reader, radio frequency ID interrogator, or other component identification sensor. In some aspects, a cassette 602 can include a first identification component, such as a bar code, and the user interface 1130 can include a reader that is configured to read and identify the first identification component. FIG. 5e shows an additional detailed view of cassette 602, including the location of secondary chamber 1150, which can be used is additional cell culture volume is required, as well as harvesting chamber 1152, which can be used to recover the final cell culture as produced herein.

In exemplary embodiments, as shown in FIG. 4f, cell culture chamber 610 further comprises at least one of: a distal port 620 configured to allow for the removal of air bubbles from the cell culture chamber and/or as a recirculation port; a medial port 622 configured to function as a recirculation inlet port; and a proximal port 624 configured to function as a drain port for cell removal.

In still further embodiments, provided herein is cassette 602 for use in an automated cell engineering system 600, comprising cell culture chamber 610 for carrying out activation, transduction and/or expansion of an immune cell culture having a chamber volume that is configured to house an immune cell culture and a satellite volume 630 for increasing the working volume of the cell culture chamber by providing additional volume for media and other working fluids without housing the immune cell culture (i.e., satellite volume does not contain any cells). Suitably, the satellite volume is fluidly connected to the cell culture chamber such that media is exchanged with the culture chamber without disturbing the immune cell culture. In exemplary embodiments, satellite volume is a bag, and in other embodiments, satellite volume is a non-yielding chamber. In embodiments, the satellite volume is between about 0.50 ml and about 300 ml, more suitably between about 150 ml and about 200 ml. FIG. 4d-4e show the position of a satellite volume 630 in cassette 602.

FIG. 4g shows a schematic illustrating the connection between cell culture chamber 610, and satellite volume 630. Also illustrated in FIG. 4g are the positioning of various sensors (e.g., pH sensor 650, dissolved oxygen sensor 651), as well as sampling/sample ports 652 and various valves (control valves 653, bypass check valves 654), as well as one or more fluidic pathways 640, suitably comprising a silicone-based tubing component, connecting the components. As described herein, use of a silicone-based tubing component allows oxygenation through the tubing component to facilitate gas transfer and optimal oxygenation for the cell culture. Also show in FIG. 4g is the use of one or more hydrophobic filters 655 or hydrophilic filters 656, in the flow path of the cassette, along with pump tube 657 and bag/valve module 658.

In embodiments, satellite volume 630 is further configured to allow media removal without loss of cells of the immune cell culture. That is, the media exchange between the satellite volume and the cell culture chamber is performed in such a manner that the cells are not disturbed and are not removed from the cell culture chamber.

In additional embodiments, as shown in FIG. 4g, cassette 602 suitably further includes a crossflow reservoir 632 for holding additional media, etc., as needed. Suitably, the crossflow reservoir has a volume of between about 0.50 ml and about 300 ml, more suitably between about 100 ml and about 150 ml.

In some embodiments, the cell engineering system includes a plurality of chambers. In further embodiments, each of the activating, transducing, expanding, concentrating, and harvesting steps of the method for cells described herein is performed in a different chamber of the plurality of chambers of the cell engineering system. In some embodiments, the cells are substantially undisturbed during transfer from one chamber to another. In other embodiments, the steps of the method are performed in the same chamber of the cell engineering system, and the cell engineering system automatically adjusts the chamber environment as needed for each step of the method. Thus, further allows for the cells to not be disturbed during the various steps.

Yields from genetically modified immune cell production, including CAR T cell production, may be influenced by activation and transduction efficiency, as well as growth conditions of the cells. Activation efficiency can improve with more stable contact between the cells and the activation reagent. Movement of the cells throughout the culture vessel may lead to an uneven distribution of the cells, and thus create localized effects when activation reagent is added to the cell culture chamber. In contrast to a flexible culture bag, cells grown in a non-yielding chamber remain undisturbed during the activation process, which may contribute to a higher activation efficiency.

Also provided herein are methods for automated production of a genetically modified immune cell culture, the method performed by a cell engineering system, comprising activating an immune cell culture with an activation reagent to produce an activated immune cell culture in a first chamber of the cell engineering system, transducing the activated immune cell culture. In exemplary methods, the transducing comprises transferring the activated immune cell culture from the first chamber to an electroporation unit, electroporating the activated immune cell culture with a vector, to produce a transduced immune cell culture, and transferring the transduced immune cell culture to a second chamber of the cell engineering system (see U.S. patent application Ser. No. 16/119,618, filed on Sep. 1, 2017, the contents of which are incorporated by reference herein in their entirety).

The methods further include expanding the transduced immune cell culture, concentrating the expanded immune cell culture of, and harvesting the concentrated immune cell culture of (d) to produce a genetically modified cell culture.

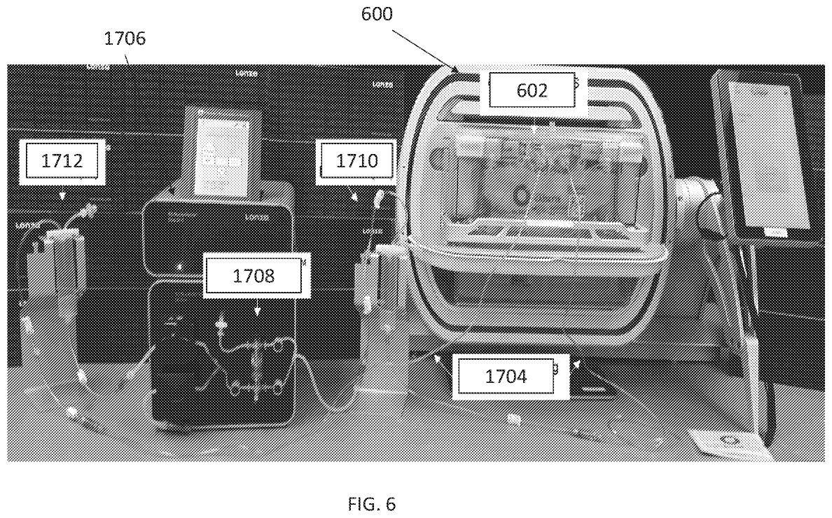

For example, as shown in FIG. 6, an activated immune cell culture is transferred, e.g., via connection tubing 1704, from cassette 602 of a cell engineering system 600 to an electroporation unit 1706. Electroporation unit 1706 suitably includes an electroporation cartridge 1708, which holds the cell culture during the electroporation process. Following the electroporation process, the transduced immune cell culture is transferred back, via connection tubing 1704, to cell engineering system 600. FIG. 6 also shows the use of two optional reservoirs 1710 and 1712, which are used to hold the cell culture prior to and after electroporation, to help in the transfer between the cell engineering system and the electroporation unit as a result of different pump speeds, required pressures and flow rates. However, such reservoirs can be removed and the cell culture transferred directly from cell engineering system 1702 to electroporation unit 1706.

In exemplary embodiments, the cell engineering systems described herein comprise a plurality of chambers, and wherein each of the steps of the various method described herein are performed in a different chamber of the plurality of chambers of the cell engineering system, each of the activation reagent, the vector, and cell culture medium are contained in a different chamber of the plurality of the chambers prior to starting the method, and wherein at least one of the plurality of chambers is maintained at a temperature for growing cells (e.g., at about 37° C.) and at least one of the plurality of chambers is maintained at a refrigerated temperature (e.g., at about 4-8° C.).

In embodiments, the monitoring includes monitoring with a temperature sensor, a pH sensor, a glucose sensor, an oxygen sensor, a carbon dioxide sensor, and/or an optical density sensor. Accordingly, in some embodiments, the cell engineering system includes one or more of a temperature sensor, a pH sensor, a glucose sensor, an oxygen sensor, a carbon dioxide sensor, and/or an optical density sensor. In additional embodiments, the cell engineering system is configured to adjust the temperature, pH, glucose, oxygen level, carbon dioxide level, and/or optical density of the cell culture, based on the pre-defined culture size. For example, if the cell engineering system detects that the current oxygen level of the cell culture is too low to achieve the necessary growth for a desired cell culture size, the cell engineering system will automatically increase the oxygen level of the cell culture by, e.g., introducing oxygenated cell culture media, by replacing the cell culture media with oxygenated cell culture media, or by flowing the cell culture media through an oxygenation component (i.e., a silicone tubing). In another example, if the cell engineering system detects that the current temperature of the cell culture is too high and that the cells are growing too rapidly (e.g., possible overcrowding of the cells may lead to undesirable characteristics), the cell engineering system will automatically decrease the temperature of the cell culture to maintain a steady growth rate (or exponential growth rate, as desired) of the cells. In still further embodiments, the cell engineering system automatically adjusts the schedule of cell feeding (i.e., providing fresh media and/or nutrients to the cell culture) based on the cell growth rate and/or cell count, or other monitored factors, such as pH, oxygen, glucose, etc. The cell engineering system may be configured to store media (and other reagents, such as wash solutions, etc.) in a low-temperature chamber (e.g., 4° C. or −20° C.), and to warm the media in a room temperature chamber or a high-temperature chamber (e.g., 25° C. or 37° C., respectively) before introducing the warmed media to the cell culture.

### Automated Process Control Systems

Automated process control systems, as discussed herein, may interact with, receive inputs from, provide inputs to, and otherwise provide all aspects of control of one or more automated cell engineering systems 600.

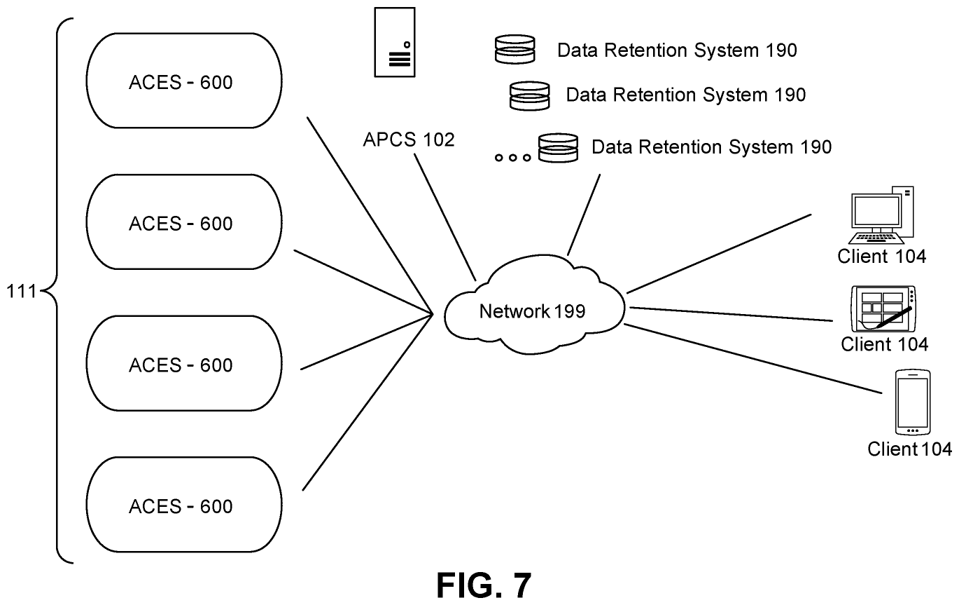

FIG. 7 illustrates an automated process control system controlling an installation of automated cell engineering system(s). In FIG. 7, an embodiment of a network environment is depicted. The network environment may include one or more automated process control system (APCS) 102 in communication with one or more automated cell engineering systems (ACES) 600, one or more data retention systems 190, one or more clients 104, via one or more networks 199. The automated cell engineering system 600 may be arranged in an automated cell engineering system installation 111, also referred to herein as an automated cell engineering system bank.

The automated cell engineering system 600 illustrated in FIG. 7 may, in an embodiment, be a Cocoon™ system as described herein. In further embodiments, the automated cell engineering system 600 may be any automated cell engineering system capable of interacting with a computing environment as described herein. As discussed above, automated cell engineering systems consistent with embodiments hereof may collect, record, and store various types of data and information. Such data and information may be stored locally, within a computer memory of the automated cell engineering system 600.

Data and information stored by an automated cell engineering system 600 may include the following information. As used herein, “automated cell engineering system data” refers to any and all data that may be recorded and stored on or in a memory of an automated cell engineering system 600. Automated cell engineering system data may be stored in any suitable data format, and may be sortable by production batch, production date, or any other suitable parameter. “Process information,” as used herein, refers to information about variables and parameters of cell culture processing, including, for example, one or more of temperature information, pH information, glucose concentration information, oxygen concentration information, component or patient identification information and optical density information, from the automated cell engineering system. Production information, as used herein, may refer to information about cell culture growth, including one or more of number of cells, cell characteristics, % transformed, etc. Control information history, as used herein, refers to information and data about user actions taken within the system. Control information history may include data about actions and about users that took such actions. Control information history may include data and information about control actions taken by a user, e.g., process parameter adjustments, as well as physical actions taken by a user in interacting directly with the automated cell engineering system 600. “Notification information,” as used herein, refers to information about notifications, alarms, alerts, and other messages directed to various users of the system. Each of the above described data and/or information may be stored as full batch records (i.e., all data pertaining to a particular cell growth batch), collective databases, data extracts (i.e., selected portions of data). Each of the above described data and/or information may be accessed in near-real time by automated process control systems 102 discussed herein.

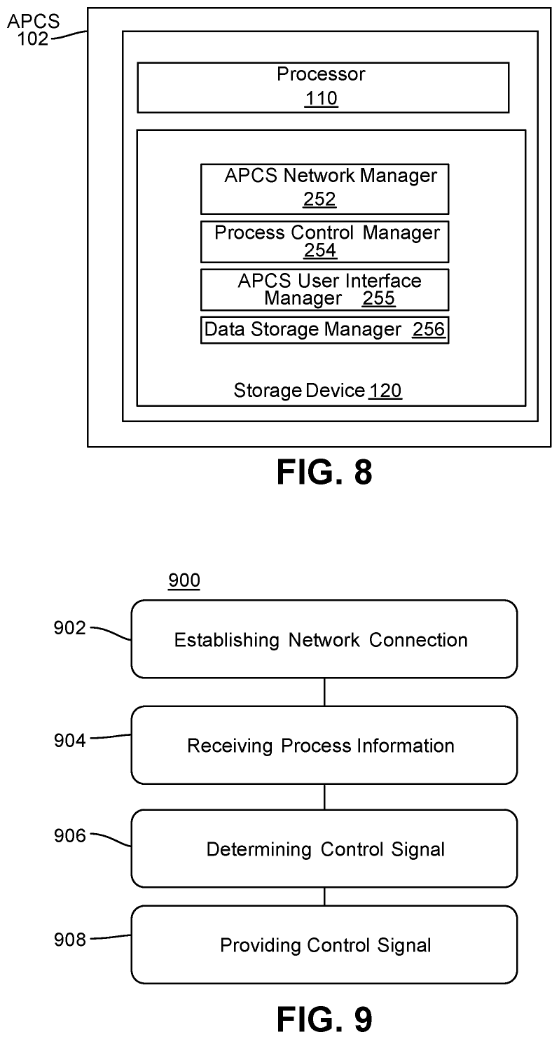

The automated process control system 102 may be configured as a server (e.g., having one or more server blades, processors, etc.), a personal computer (e.g., a desktop computer, a laptop computer, etc.), a smartphone, a tablet computing device, and/or other device that can be programmed to interface with an automated cell engineering system 600. In an embodiment, any, or all of the functionality of the automated process control system 102 may be performed as part of a cloud computing platform. The automated process control system 102 is further discussed below with respect to FIG. 8.

The one or more clients 104 may be configured as a personal computer (e.g., a desktop computer, a laptop computer, etc.), a smartphone, a tablet computing device, and/or other device that can be programmed with a user interface for accessing the automated cell engineering system 600 and/or the automated process control system 102. In embodiments, the one or more clients 104 may be include multiple devices, such as a facility management system including a network of servers, workstations, additional clients, etc. In embodiments, the automated process control system 102 and a client 104 may reside within a single system, such as a laptop, desktop, tablet, or other computing device with a user interface. A suitably configured client 104 may provide a user with access to all of the functionality of the automated process control system 102 as described herein.

The network environment depicted in FIG. 7 represents an example embodiment of an automated process control system 102 configured to control an automated cell engineering system installation 111. Although depicted as connected via network 199, any suitable series of individual or network connections may be employed to permit an automated process control system 102 to control an automated cell engineering system installation 111 and access required resources such as various data retention systems 190.

The network 199 may be connected via wired or wireless links Wired links may include Digital Subscriber Line (DSL), coaxial cable lines, ethernet, or optical fiber lines. Wireless links may include Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, NFC, SigFox, LoRa, Random Phase Multiple Access (RPMA), Weightless-N/P/W, an infrared channel or a satellite band. The wireless links may also include any cellular network standards to communicate among mobile devices, including standards that qualify as 2G, 3G, 4G, or 5G. Wireless standards may use various channel access methods, e.g., FDMA, TDMA, CDMA, or SDMA. In some embodiments, different types of data may be transmitted via different links and standards. In other embodiments, the same types of data may be transmitted via different links and standards. Network communications may be conducted via any suitable protocol, including, e.g., http, tcp/ip, udp, ethernet, ATM, etc.

The network 199 may be any type and/or form of network. The geographical scope of the network may vary widely and the network 199 can be a body area network (BAN), a personal area network (PAN), a local-area network (LAN), e.g., Intranet, a metropolitan area network (MAN), a wide area network (WAN), or the Internet. The topology of the network 199 may be of any form and may include, e.g., any of the following: point-to-point, bus, star, ring, mesh, or tree. The network 199 may be of any such network topology as known to those ordinarily skilled in the art capable of supporting the operations described herein. The network 199 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv4 and IPv4), or the link layer. The network 199 may be a type of broadcast network, a telecommunications network, a data communication network, or a computer network.

The data retention systems 190 may include any type of computer readable storage medium (or media) and/or a computer readable storage device. Such computer readable storage medium or device may be configured to store and provide access to data. Examples of computer readable storage medium or device may include, but is not limited to, an electronic storage device, a magnetic storage device, an optical storage device, an electromagnetic storage device, a semiconductor storage device, or any suitable combination thereof, for example, such as a computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick.

FIG. 8 illustrates an automated process control system consistent with embodiments hereof. The automated process control system 102 includes one or more processors 110 (also interchangeably referred to herein as processors 110, processor(s) 110, or processor 110 for convenience), one or more storage device(s) 120, and/or other components. In other embodiments, the functionality of the processor may be performed by hardware (e.g., through the use of an application specific integrated circuit (“ASIC”), a programmable gate array (“PGA”), a field programmable gate array (“FPGA”), etc.), or any combination of hardware and software. The storage device 120 includes any type of non-transitory computer readable storage medium (or media) and/or non-transitory computer readable storage device. Such computer readable storage media or devices may store computer readable program instructions for causing a processor to carry out one or more methodologies described here. Examples of the computer readable storage medium or device may include, but is not limited to an electronic storage device, a magnetic storage device, an optical storage device, an electromagnetic storage device, a semiconductor storage device, or any suitable combination thereof, for example, such as a computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, but not limited to only those examples.

The processor 110 is programmed by one or more computer program instructions stored on the storage device 120 representing software protocols. For example, the processor 110 is programmed by an automated process control system (apcs) network manager 252, a process control manager 254, an automated process control system (apcs) interface manager 255, and an automated process control system (apcs) data storage manager 256. It will be understood that the functionality of the various managers as discussed herein is representative and not limiting. Additionally, the storage device 120 may act as a data retention system 190 to provide data storage. As used herein, for convenience, the various “managers” will be described as performing operations, when, in fact, the managers program the processor 110 (and therefore the automated process control system 102) perform the operation.

The various components of the automated process control system 102 work in concert to provide control of one or more automated cell engineering systems 600 or automated cell engineering system installation 111 and to provide an interface for a user or other system to interface with one or more automated cell engineering systems 600 or automated cell engineering system installation 111.

The apcs network manager 252 is a software protocol operating on the automated process control system 102. The apcs network manager 252 is configured to establish a network communication between the automated process control system 102, automated cell engineering systems 600, automated cell engineering system installation 111, data retention systems 190, and clients 104. The established communications pathway may utilize any appropriate network transfer protocol and provide for one way or two way data transfer. The apcs network manager 252 may establish as many network communications as required to communicate with one or more automated cell engineering system 600 and other components of the automated cell engineering system installation 111, data retention systems 190, clients 104, etc.

The apcs network manager 252 allows for the sending and receiving, with one or more automated cell engineering system 600, of instructions, process parameters, automated cell engineering system data, cell growth protocols, software upgrades, user authentication information, and production orders. Production orders, as used herein, refers to orders for the production of one or more cell cultures. Production orders may include information about cell culture growth protocols to be used, initial information about cells prior to initiation of a cell culture growth protocol, and other required information for the production of a cell culture. The apcs network manager 252 may facilitate the receiving of process information from the automated cell engineering system 600, including, but not limited to one or more of temperature information, pH information, glucose concentration information, oxygen concentration information, carbon dioxide concentration information, optical density information, magnetic state information, and any other process information collected by the one or more automated cell engineering systems 600 as discussed herein. The apcs network manager 252 may also facilitate the receiving of production information from the automated cell engineering system 600, including one or more of number of cells, cell characteristics, % transformed, etc. recorded over time.

The apcs network manager 252 further facilitates the sending and receiving, with one or more clients 104, automated cell engineering system status information, data including full batch records, data extracts, real-time data, and archived data, data analysis produced and/or provided by the automated process control system 102, and compliance and/or reporting information. The apcs network manager 252 further facilitates the sending and receiving of archival data to one or more data retention systems 190.

The process control manager 254 is a software protocol operating on the automated process control system 102. The process control manager 254 is configured to provide one or more control signals to one or more automated cell engineering system 600. The control signals provided by the process control manager 254 are configured to cause an adjustment of one or more process parameters of the automated cell engineering system 600. As used herein, “process parameters” refers to any parameter or variable of the production process that can be adjusted by a user through automated process control system 102. Process parameters include but are not limited to gas concentration, media conditions, temperature, pH, waste and nutrient concentrations, and media flow rates. Determination of the control signals may be based on process information received by the apcs network manager 252. Determination of the control signals may further be based on the production information received by the apcs network manager 252.

Control signals provided by the process control manager 254 may be used to initiate and/or control any process that an automated cell engineering system 600 described herein is capable of Such processes may include, but are not limited to all steps, processes, and actions related to fractionation, cell seeding, activation, transduction, electroporation, feeding, selection, harvest, washing, concentration, formulation, etc.

In embodiments, the process control manager 254 may operate to update, alter, and/or adjust process parameters of the one or more automated cell engineering system 600 to which the automated process control system 102 is connected via one or more control signals, as discussed further below. Any update performed by the process control manager 254 may be performed automatically, without user supervision, responsive to information collected and according to cell culture growth protocols.

In embodiments, updates may require user authorization. In such embodiments, the process control manager 254 may send a request to one or more authorized users to approve a process parameter alteration. Such requests may be sent directly to the screen or to an inbox of a client 104 connected to the automated process control system 102 and/or may be sent via alternative communication means such as e-mail, text message, or voice message. In some embodiments, the process control manager 254 may interpret a lack of response to an authorization request, after a certain time period, as a denial of the request. In some embodiments, the process control manager 254 may interpret a lack of response to an authorization request, after a certain time period, as an approval of the request.

Process parameters of the automated cell engineering system 600 that may be adjusted by the process control manager 254 include one or more gas concentration, media conditions, temperature, pH, waste and nutrient concentrations, and media flow rates, electroporation conditions, transduction conditions, etc. Adjustment of these various process parameters may be performed based on the process information received from the automated cell engineering system 600. As discussed above, an automated cell engineering system 600 is an autonomous system and may not require external control to maintain process parameters at programmed levels. The process control manager 254 may, however, be configured to adjust the programmed levels for various process parameters based on process information. The process control manager 254 may operate to perform any or all process control operations described herein on an on-going, real-time, or recurring basis.

For example, process information, such as temperature information, pH information, glucose concentration, component or patient identification information, oxygen concentration information and/or optical density information may show that one or more of these values differs from an expected or programmed value despite autonomous control. The process control manager 254 may therefore adjust an appropriate process parameter in response.

In another example, the process control manager 254 may be used to alter process parameters in accordance with a cell culture growth protocol (i.e., a desired increase in cell volume, transduction time, growth rate changes, etc.). A cell culture growth protocol may require updating to process parameters during a cell engineering process. The process control manager 254 may implement such an adjustment.

In another example, the process control manager 254 may be used to alter process parameters in accordance with a cell culture growth protocol update. A cell culture growth protocol may be updated or otherwise altered during a cell engineering process. Such an update may therefore require a process parameter update to be implemented by the process control manager 254.

In yet another example, the process control manager 254 may update process parameters in a first automated cell engineering system 600 according to production information received from a second automated cell engineering system 600. For example, a first cell engineering process in a first automated cell engineering system 600 may be exceeding expectations for production levels and a second cell engineering process in a second automated cell engineering system 600 may have its process parameters adjusted to reduce or alter production.

In still another example, cell production in an automated cell engineering system 600 may vary from levels expected based on initial process parameters. Production information may show that cell production is greater than or less than expected. Accordingly, process parameters may be adjusted by the process control manager 254 responsive to the production information.

In embodiments, the process control manager 254 provides a process monitoring function. The process control manager 254 may be configured to access any and all information measured, produced, and/or stored by the automated cell engineering system 600. The process control manager 254 may further be configured to provide any of such information to a user via the apcs user interface manager 255.

In further embodiments, the process control manager 254 may be equipped for automated cell engineering system 600 diagnostics. Accordingly, the process control manager 254 may review system performance, including process information, process parameters, user control history, and production information and compare these information against calibrated levels and/or other benchmarks to determine that an automated cell engineering system 600 is operating within specification.

The apcs user interface manager 255 is a software protocol operating on the automated process control system 102. The apcs user interface manager 255 is configured to provide a user interface to allow user interaction with the automated process control system 102. The apcs user interface manager 255 is configured to receive input from any user input source, including but not limited to touchscreens, keyboards, mice, controllers, joysticks, voice control. The apcs user interface manager 255 is configured to provide a user interface, such as a text based user interface, a graphical user interface, or any other suitable user interface. The apcs user interface manager 255 is configured to use the apcs network manager 252 to provide such user interface services through one or more clients 104. The apcs user interface manager 255 may be configured to provide different user interface services depending on a type of client device. For example, a laptop or desktop computer may be provided with a user interface including a full suite of interface options, while a smartphone or tablet may be provided with a user interface limited to status updates.

The apcs user interface manager 255 is configured to provide user authentication services. Users may be authenticated via, for example, passwords, biometric scanning (retina scans, fingerprints, voice prints, facial recognition, etc.), key cards, token access, and any other suitable means of user authentication. User authentication services may be provided to control access to one or more automated cell engineering system 600.

In embodiments, one or more users may be provided full access to all functionality, process information, and/or production information of an automated cell engineering system 600 or automated cell engineering system installation 111. One or more users may be provided with limited access to functionality, process information, and/or production information of an automated cell engineering system 600 or all automated cell engineering systems within an automated cell engineering system installation 111. One or more users may be provided with full access to a limited portion of automated cell engineering systems 600 within an automated cell engineering system installation 111. In some embodiments, one or more users may be provided with “read only” access that permits viewing of process information, production information, etc., but does not permit any adjustments to process parameters. Further, one or more users may be provided with full or limited access to archived data. Access controls may be determined according to user identity, user function, user job identity, and any other suitable criteria.

In embodiments, the apcs user interface manager 255 may provide one or more users with access to any or all process and/or production information about one or more automated cell engineering system 600 via a user interface. The apcs user interface manager 255 may permit a user to perform various tasks on one or more automated cell engineering system 600 within an automated cell engineering system installation 111. For example, the apcs user interface manager 255 may permit a user to adjust or control one or more process parameters directly. In another example, the apcs user interface manager 255 may permit a user to update a cell culture growth protocol. In another example, the apcs user interface manager 255 may permit a user to adjust a process goal and the autonomous automated cell engineering system 600 or process control manager 254 may automatically adjust process parameters to achieve the specified goal.

In embodiments, apcs user interface manager 255 is configured to provide user training, tutorials, and assessments for automated cell engineering system 600. The apcs user interface manager 255 may, in conjunction with the automated cell engineering system 600, enter a training mode. In a training mode, the apcs user interface manager 255 may provide a user with operational instructions for carrying out various cell engineering tasks. The apcs user interface manager 255 may operate in conjunction with the automated cell engineering system 600, for example, by causing the automated cell engineering system 600 to perform operations as a user works through a training mode. In further embodiments, the apcs user interface manager 255 may cause the automated cell engineering system 600 to also present the user with text prompts, visual highlights, and other cues to assist training

The apcs data storage manager 256 is a software protocol operating on the automated process control system 102. The apcs data storage manager 256 is configured to access one or more automated cell engineering system 600 to receive and/or retrieve automated cell engineering system data. Automated cell engineering system data may include, for example, production information, which may be obtained in near real time, archived data, and/or data extracts, as well as process information and process parameter information and any other information or data generated by an automated cell engineering system 600. The apcs data storage manager 256 is further configured to access one or more data retention systems 190 to store and/or receive automated cell engineering system data stored in the data retention system 190.

The apcs data storage manager 256 may provide data to a user via the automated process control system interface manager 255. In embodiments, the apcs data storage manager 256 is further configured to provide access tools to the user to manage, access, and analyze automated cell engineering system data. For example, the apcs data storage manager 256 may be configured to generate reports, collate automated cell engineering system data, cross-reference automated cell engineering system data, populate databases with automated cell engineering system data, etc.

In embodiments, the apcs data storage manager 256 may provide data retention capabilities. The apcs data storage manager 256 is configured to receive new batch record data from each automated cell engineering system 600 connected to the automated process control system 102 at a configurable interval—e.g., every ten seconds, every thirty seconds, minute, every five minutes, every ten minutes, every hour, etc. The configurable interval may be adjusted according to a cell culture growth protocol. For example, critical processes that require close monitoring may have shorter intervals while non-critical processes may have longer intervals. In embodiments, the apcs data storage manager 256 may be further configured to receive new recorded data from one or more automated cell engineering systems 600 according to the occurrence of events at the associated automated cell engineering systems 600. In further embodiments, the apcs data storage manager 256 is further configured to receive new recorded data at regular configurable intervals and according to the occurrence of events. As the new batch record data is received from each automated cell engineering system 600, the apcs data storage manager 256 stores the new data in a local database associated with the automated cell engineering system 600 on the storage device 120. In embodiments, data from one or more automated cell engineering systems 600 may be stored in the same database. Each automated cell engineering system 600 may be associated with a specific database on the storage device 120. When a new set of batch record data is generated on an automated cell engineering system 600, e.g., due to initiation of a new cell culture growth protocol, a new database on automated process control system 102 may be generated accordingly. In embodiments, a previously created database may be used to store information from the initiation of a new cell culture growth protocol. If required, for example, because a cell culture is transferred from one automated cell engineering system 600 to another automated cell engineering system 600, the appropriate batch record data may be transferred as well, permitting the new automated cell engineering system 600 to access all required information for that particular cell culture.

In embodiments, the apcs data storage manager 256 may provide enhanced data retention capabilities. At regular intervals as required, the batch record databases stored locally on the storage device 120 of the automated process control system 102 may be transferred to one or more data retention systems for archival purposes. The newly archived data may be verified by the apcs data storage manager 256. In the case of a failure to verify data archived in the one or more data retention systems 190, the archival process may be repeated based on the batch record database stored on the storage device 120 and/or based on receiving the data again from the automated cell engineering system 600. After verification of data archival, deletion of data on the automated cell engineering system 600 and/or the local data copy on the storage device 120 may be scheduled for the future or may be performed.

In embodiments, the apcs data storage manager 256 may be configured to store and manage data records in compliance with Federal Regulations such as 21 C.F.R. part 11. For example, apcs data storage manager 256 may implement user access controls, data validation checks, archival backups, data reproductions, data auditing, and other processes in compliance with Federal Regulations.

As discussed above, the various components of the automated process control system 102 may work in concert to provide control of one or more automated cell engineering systems 600 or an automated cell engineering system installations 111 and to provide an interface for a user or other system to interface with one or more automated cell engineering systems 600 or an automated cell engineering system installation 111. In embodiments, the one or more automated cell engineering systems 600 or automated cell engineering system installation 111 may be controlled through a combination of local direct control of each individual automated cell engineering system 600 and control via the automated process control system 102. All of the process control functionality of the automated cell engineering systems 600, as described above with respect to FIGS. 1-6, may be conducted either through direct interaction with an automated cell engineering system 600 or via the automated process control system 102, in any combination. Conversely, in further embodiments, all of the functionality of the automated process control system 102, as discussed with respect to FIG. 8, may be conducted either through direct interaction with an automated cell engineering system 600 or via the automated process control system 102, in any combination. In further embodiments, a processor of an automated cell engineering system 600 may be configured to run any of the software protocols described herein with respect to the automated process control system 102 (e.g., the apcs network manager 252, the process control manager 254, the apcs user interface manager 255, and the data storage manager 256) and, therefore, to operate as both an automated cell engineering system 600 and an automated process control system 102.

For example, in embodiments. process control steps, such as those described with respect to FIGS. 1-6, may be carried out directly via operator interaction with an automated cell engineering system 600. An operator may, for example, directly access the automated cell engineering system 600 to monitor on-going processes and initiate new processes at the appropriate time. User identification and authorization functionality may be carried out at the automated cell engineering system 600 to ensure appropriate access. In such embodiments, the automated process control system 102 may collect and archive data (e.g., process information, production information, and control information) from ongoing processes in the automated cell engineering system 600, may perform system monitoring to ensure proper function of the automated cell engineering system 600, may adjust general parameters and settings within the automated cell engineering system 600, and perform any other functions to ensure the proper function and monitoring of the automated cell engineering system 600. In such an embodiment, the automated process control system 102 performs oversight of the one or more automated cell engineering systems 600 while permitting local process control to occur directly at the automated cell engineering system 600. Due to the monitoring function, the automated process control system 102 may be configured to provide alerts, notifications, or other prompts when local control of the automated cell engineering system 600 deviates from expected or planned process parameters.

In further embodiments, the automated process control system 102 may be employed only for data gathering and archival purposes without providing any monitoring or control functions. In further embodiments, the automated process control system 102 may provide coordination between the multiple automated cell engineering systems 600 of an installation. For example, the automated process control system 102 may supply process information to the automated cell engineering system 600 for the use of an operator to access and execute locally via direct interface with the automated cell engineering system 600. A customer request for several production orders may, for example, be allocated by the automated process control system 102 across several automated cell engineering systems 600 and then be executed by local operators at each individual automated cell engineering systems 600.

The above described breakdowns of workflows as performed via an automated cell engineering system 600 or via an automated process control system 102 are by way of example only. Any combination of the automated cell engineering system 600 functionality and the automated process control system 102 functionality as described herein may be employed in the operation of the automated cell engineering systems 600.

FIG. 9 is a flow chart showing a process 900 of controlling an automated cell engineering system 600. The process 900 is performed on a computer system having one or more physical processors programmed with computer program instructions that, when executed by the one or more physical processors, cause the computer system to perform the method. The one or more physical processors are referred to below as simply the processor. In embodiments, the various operations of the process 900 are carried out via the automated process control system 102, via direct interface with the automated cell engineering system 600, and/or via any combination as described herein. The automated process control system 102 represents an example of a hardware and software combination configured to carry out process 900, but implementations of the process 900 are not limited to the hardware and software combination of the automated process control system 102. Additional details regarding each of the operations of the method may be understood according to the description the automated process control system 102, as described above.

In an operation 902, process 900 includes establishing a network connection with an automated cell engineering system. A network connection between an automated process control system as described herein and an automated cell engineering system as described herein may be established via any suitable network transmission protocol or protocol suite, including, e.g., http, TCP/IP, LAN, WAN, WiFi, etc.

In an operation 904, process 900 includes receiving process information from the automated cell engineering system 600. The automated process control system may receive process information, including, for example, one or more of temperature information, pH information, glucose concentration information, oxygen concentration information, component or patient identification information, and optical density information from the automated cell engineering system.

In an operation 906, process 900 includes determining a control signal to adjust one or more process parameters of the automated cell engineering system. The control signal is determined by the automated process control system and may be responsive to the process information received. The control signal determination may further be responsive to production information received from the automated cell engineering system, to cell culture growth protocol updates or alterations, and/or to user initiated updates or alterations. The control signal may further be responsive to each of these factors.

In an operation 908, process 900 includes providing the control signal to the automated cell engineering system. The control signal, determined by the automated process control system, may be provided to the automated cell engineering system via the network connection. Responsive to receiving the control signal, the automated cell engineering system may adjust one or more process parameters to achieve alterations in production and/or process conditions.

As discussed above, the various functional aspects of the process 900 may be performed either by the automated process control system 102 or via direct interface with the automated cell engineering system 600. For example, the networking and process information operations 902 and 904 may provide, via a network, process information to the automated cell engineering system 600 while a local operator, via direct interface with controls of the automated cell engineering system 600, may cause the generation and provision of the control signal to adjust the process parameters within the automated cell engineering system 600.

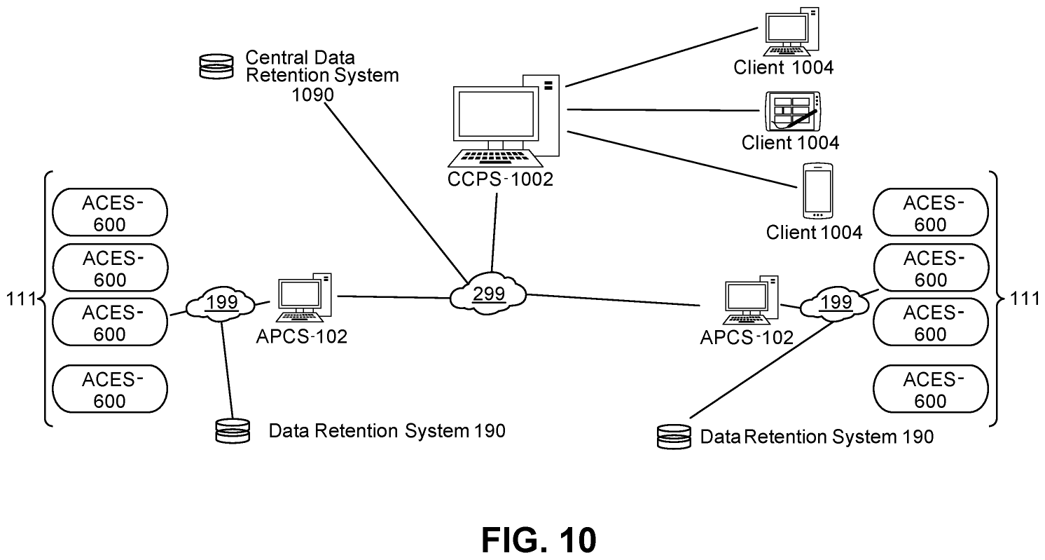

FIG. 10 illustrates a central control process system controlling multiple automated process control system installations. A central control process system 1002 is provided to interface with one or more automated process control systems 102, each of which is connected to an automated cell engineering system installation 111 and a data retention system 190 via a network 199. The central control process system 1002 is configured to interface with each automated process control system 102 via the network 299 and may additionally access a central data retention system 1090. Users may access the central control process system 1002 via direct interaction with the central control process system 1002 and/or via one or more client 1004.

The one or more clients 1004 may be configured as a personal computer (e.g., a desktop computer, a laptop computer, etc.), a smartphone, a tablet computing device, and/or other device that can be programmed with a user interface for accessing central control process system 1002. In embodiments, the central control process system 1002 and a client 1004 may reside within a single system, such as a laptop, desktop, tablet, or other computing device with a user interface. A suitably configured client 1004 may provide a user with access to all of the functionality of the central control process system 1002 as described herein.

The network 299 may have any or all of the characteristics discussed above with respect to network 199. In embodiments, network 199 and network 299 may be the same network. Each automated process control system 102 and its associated systems and components corresponds to the automated process control system 102 described above with respect to FIGS. 7 and 8.

The central control process system 1002 is configured to monitor, update, and interact with one or more local automated process control systems 102. The central control process system 1002 may, for example, push software updates, update and manage cell culture growth protocols, manage user access, conduct second eye monitoring of automated cell engineering system 600, conduct quality control activities, etc., as described herein. The central control process system 1002 may coordinate the activities and operations of multiple automated cell engineering system installations 111 via their associated automated process control systems 102.

The central control process system 1002 is connected to a central data retention system 1090. The central data retention system 1090 is a computer information storage device and shares any or all characteristics described above with respect to data retention systems 190. Although depicted as connected to central control process system 1002 via network 299, the central data retention system 1090 may also be collocated with the central control process system 1002 (e.g., the central control process system 1002 and central data retention system 1090 may share an enclosure and/or may share a computer readable memory device), and may also be directly connected to central control process system 1002.

In further embodiments, the central control process system 1002 may provide all of the functionality of an automated process control system 102 as described above and may be employed to interact with and access any automated cell engineering system 600 within the system in the same fashion as a locally associated automated process control system 102. For example, an authorized user may operate central control process system 1002 to access any specific connected automated cell engineering system installation 111 with all of the functionality and access of the associated local automated process control system 102.

In further embodiments, the central control process system 1002 may facilitate access to any automated cell engineering system 600 within the connected system by any given local automated process control system 102. For example, an authorized user at a first automated process control system 102 associated with a first automated cell engineering system installation 111 may access a second automated cell engineering system installation 111 associated with a second automated process control system 102 via the central control process system 1002. Accordingly, the networked system of central control process system 1002 and automated process control systems 102 may provide users that have appropriate authorization access and control over any automated cell engineering system 600 in the system. The central control process system 1002 may further facilitate access to the central data retention system 1090 via any automated process control system 102.

In further embodiments, any and all functionality of a central control process system 1002 may be implemented by an automated process control system 102. In still further embodiments, a central control process system 1002 and an automated process control system 102 may be implemented by the same processor or processors.

Although FIG. 10 illustrates a system including a single central control process system 1002 and two automated process control systems 102, the invention is not so limited. A networked system of automated cell engineering system installations 111 may include any number of central control process systems 1002 and automated process control systems 102.

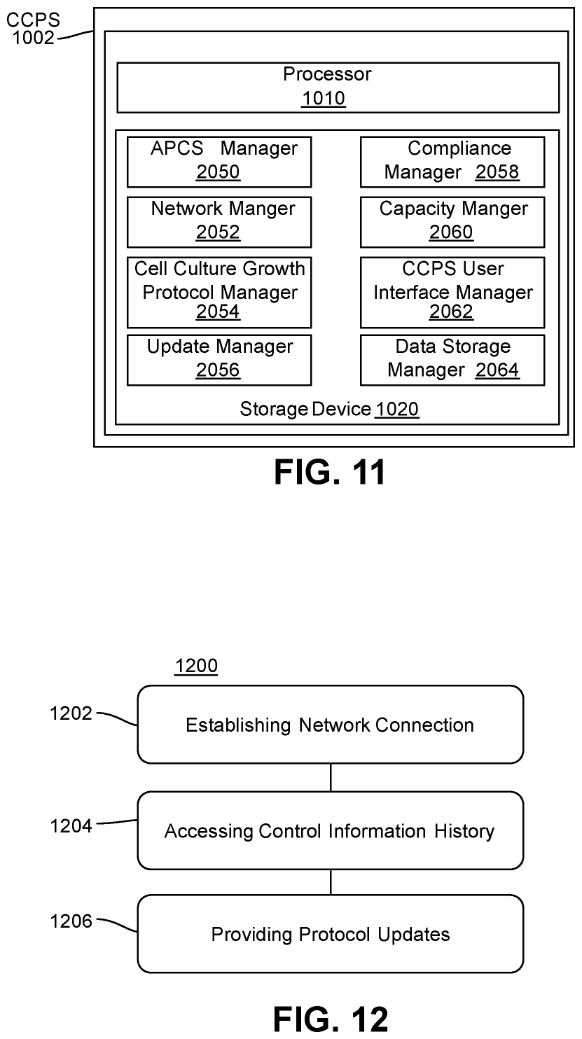

FIG. 11 illustrates a central control process system consistent with embodiments hereof. The central control process system 1002 includes one or more processors 1010 (also interchangeably referred to herein as processors 1010, processor(s) 1010, or processor 1010 for convenience), one or more storage device(s) 1020, and/or other components. In other embodiments, the functionality of the processor may be performed by hardware (e.g., through the use of an application specific integrated circuit (“ASIC”), a programmable gate array (“PGA”), a field programmable gate array (“FPGA”), etc.), or any combination of hardware and software. The storage device 1020 includes any type of non-transitory computer readable storage medium (or media) and/or non-transitory computer readable storage device. Such computer readable storage media or devices may store computer readable program instructions for causing a processor to carry out one or more methodologies described here. Examples of the computer readable storage medium or device may include, but is not limited to an electronic storage device, a magnetic storage device, an optical storage device, an electromagnetic storage device, a semiconductor storage device, or any suitable combination thereof, for example, such as a computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, but not limited to only those examples.

The processor 1010 is programmed by one or more computer program instructions stored on the storage device 1020 representing software protocols. For example, the processor 1010 is programmed by an automated process control system manager 2050, a central control process system (ccps) network manager 2052, a cell culture growth protocol manager 2054, an update manager 2056, a compliance manager 2058, a capacity manager 2060, a central control process system (ccps) user interface manager 2062, and a central control process system (ccps) data storage manager 2064. It will be understood that the functionality of the various managers as discussed herein is representative and not limiting. Additionally, the storage device 1020 may act as the central data retention system 1090 to provide data storage. As used herein, for convenience, the various “managers” will be described as performing operations, when, in fact, the managers program the processor 1010 (and therefore the central control process system 1002) to perform the operation.

The various components of the central control process system 1002 work in concert to provide control of one or more automated process control systems 102, automated cell engineering systems 600, and/or automated cell engineering system installations 111 and to provide an interface for a user or other system to interface with these.

The automated process control system manager 2050 is a software protocol in operation on central control process system 1002. The automated process control system manager 2050 is configured to provide the central control process system 1002 with any and all of the functionality of an automated process control system 102 with respect to any automated cell engineering system 600 or automated cell engineering systems installation 111 to which the central control process system 1002 is connected via network or other connection. Accordingly, the automated process control system manager 2050 can perform and provide all of the functions described herein with respect to the apcs network manager 252, the process control manager 254, the apcs user interface manager 255, and the apcs data storage manager 256.

For example, the automated process control system manager 2050 is configured to provide production control and management functionality to the central control process system 1002. Whereas a user of an automated process control system 102 may create production orders and manage cell production across one automated cell engineering system 600 or an automated cell engineering system installation 111, a user of a central control process system 1002 may create production orders and manage cell production across multiple automated cell engineering systems 600 and automated cell engineering system installations 111 concurrently.

The automated process control system manager 2050 is configured to access control information history of one or more of the automated process control systems 102 to which a connection has been established. Control information history includes information and/or data about automated cell engineering system 600 performance. Such information includes records of control signals, process parameters, process information, and production information recorded over time. Accordingly, control information history includes detailed historical information about commands and control signals sent to one or more automated cell engineering system 600 and historical information about automated cell engineering system performance in response to such commands and control signals. Control information history further includes information and data about the autonomous function of one or more automated cell engineering system 600 and/or automated cell engineering system installation 111 within the system. Control information history may be used by central control process system 1002 to monitor, troubleshoot, update, upgrade, and otherwise control the performance of one or more automated process control system 102 and associated automated cell engineering systems 600.

The ccps network manager 2052 is a software protocol in operation on central control process system 1002. The ccps network manager 2052 is configured to establish network communications between the central control process system 1002, automated process control systems 102, central data retention system 1090, and clients 1004. The ccps network manager 2052 is thus configured to establish network connections with a plurality of automated process control systems 102, each of which controls one or more automated cell engineering system 600 or automated cell engineering system installation 111. The established communications pathway may utilize any appropriate network transfer protocol and provide for one way or two way data transfer. The ccps network manager 2052 may establish as many network communications as required to communicate with one or more automated process control system 102. In further embodiments, the ccps network manager 2052 may be configured to establish network communications with one or more automated cell engineering systems 600, automated cell engineering system installations 111, and/or data retention systems 190.

The ccps network manager 2052 allows for the sending and receiving, with one or more automated process control system 102, of instructions, data including full batch records, data extracts, near or substantially real-time data, and archived data, protocols, software upgrades, user authentication information, production orders, process information, production information, and any other data or information obtained, accessed, or stored by the automated process control systems 102. The ccps network manager 2052 further facilitates communications with the one or more clients 1004 to allow user access to the central control process system 1002 and communications with the automated process control systems 102 to permit the various other software protocols in operation on the central control process system 1002 to perform their required functions.

The cell culture growth protocol manager 2054 is a software protocol in operation on central control process system 1002. The cell culture growth protocol manager 2054 is configured to create, store, maintain, and update cell culture growth protocols. The cell culture growth protocol manager 2054 stores a plurality of cell culture growth protocols in the central data retention system 1090. The cell culture growth protocol manager 2054 further permits a user to create and update cell culture growth protocols via interaction through the ccps user interface manager 2062, discussed further below. Newly created and updated cell culture growth protocols may be pushed from the cell culture growth protocol manager 2054 to one or more automated process control system 102 as a new protocol or an update protocol for use by the automated process control system 102 in controlling an automated cell engineering system 600 or an automated cell engineering system installation 111.

In embodiments, the cell culture growth protocol manager 2054 may maintain one or more databases of cell culture growth protocols in the central data retention system 1090. Cell culture growth protocol databases may include information about which automated cell engineering systems 600 and/or automated process control systems 102 have access to certain protocols, what versions or protocols may be accessed, production information associated with various protocols and automated process control system 102. Such information may be used, for example, for quality control purposes to ensure that similar protocols are performing with similar results in different automated cell engineering system installations 111. Such information may further be used, for example, to compare production results between multiple versions of a same protocol across multiple automated cell engineering system installations 111.

In embodiments, the cell culture growth protocol manager 2054 may provide protocol development capabilities. The cell culture growth protocol manager 2054 may receive automated cell engineering system data including protocol information, process information, production information, and all other relevant data collected by one or more automated cell engineering system installations 111 associated with the central control process system 1002. The cell culture growth protocol manager 2054 may compare information obtained from the multiple automated cell engineering system installations 111 to determine factors promoting the success of cell culture growth protocols. Such factors may include, for example, the various process parameters and/or differences in cell culture growth protocols. In embodiments, the cell culture growth protocol manager 2054 may analyze the automated cell engineering system data for the purposes of identifying successful treatment protocols, troubleshooting unsuccessful treatment protocols, and developing successful treatment protocols. Developed and identified successful treatment protocols may be communicated by the cell culture growth protocol manager 2054 to the one or more automated process control system 102 associated therewith. Information regarding the troubleshooting may be communicated to automated process control systems 102 associated with the unsuccessful treatment protocols to permit an authorized user to adjust the protocols.

The update manager 2056 is a software protocol in operation on central control process system 1002. The update manager 2056 is configured to maintain records of cell engineering system software versions in use on one or more automated process control system 102 and one or more automated cell engineering system 600 to which the central control process system 1002 is connected. The update manager 2056 is further configured to provide cell engineering software updates to the one or more automated process control system 102 and the one or more automated cell engineering system 600 to which the central control process system 1002 is connected.

In embodiments, the update manager 2056 is configured to automatically push software updates to automated process control systems 102 and automated cell engineering systems 600 that require updates. In embodiments, the update manager 2056 is configured to request user authorization to provide an update. In further embodiments, the update manager 2056 is configured to notify a locally authorized user of an automated process control system 102 or automated cell engineering system 600 of the availability of a software update.

In embodiments, the update manager 2056 is configured to receive, from an automated process control system 102, a notification that no cell engineering software updates are to be provided until after a certain period of time, after a certain number of production runs, or after a specific authorized user request. Because automated cell engineering systems 600 and automated process control systems 102 may be used for conducting validated cell growth projects and experiments, it may be required to maintain usage of a specifically validated software version throughout a specific project.

The compliance manager 2058 is a software protocol in operation on central control process system 1002. The compliance manager 2058 is configured to analyze information history collected by the central control process system 1002 to determine whether one or more automated process control systems 102 and automated cell engineering systems 600 are being used in a compliant fashion. It may be checked or determined to ensure that appropriate regulations are being complied with and/or checked or determined to ensure that appropriate guidelines are being complied with. Appropriate regulations may include government regulations, such as FDA regulations. Appropriate guidelines may include corporate guidelines, ethical guidelines, best practices, and/or other guidelines instituted by an operator/owner of the central control process system 1002.

For example, the compliance manager 2058 may be used to analyze control information history to determine and/or ensure that an automated cell engineering system installation 111 associated with an automated process control system 102 is being used in an ethical manner. The control information history may be compared to the user log maintained by the apcs user interface manager 255 to determine which users are or are not using the automated cell engineering system installation 111 according to ethical guidelines. Responsive to determining that one or more user are not using the automated cell engineering system installation 111 according to ethical guidelines (or other guidelines, regulations, or best practices), the compliance manager 2058 may act through the ccps user interface manager 2062 to modify local user access to the automated process control system 102. For example, the compliance manager 2058 may restrict local user access of one or more local users based on the control information history.

The capacity manager 2060 is a software protocol in operation on central control process system 1002. The capacity manager 2060 is configured to manage capacity across the one or more automated cell engineering system installations 111 to which the central control process system 1002 is connected via network communications. The capacity manager 2060 is configured to maintain records, stored, e.g., in the central data retention system 1090, of automated cell engineering systems 600 that are or are not in use across the central control process system 1002 connected system. The capacity manager 2060 is further configured to maintain records of expected future usage of automated cell engineering systems 600 across the central control process system 1002 connected system. For example, the capacity manager 2060 may predict a future date at which an automated cell engineering system 600 will no longer be in use according to protocol and production information of the automated cell engineering system 600. In another example, the capacity manager 2060 may access production order information of an automated process control system 102 to determine how many automated cell engineering systems 600 associated with the automated process control system 102 may go into use in the future.

The capacity manager 2060 may provide to a user, via the ccps user interface manager 2062, knowledge and/or information regarding automated cell engineering system 600 capacity at various automated cell engineering system installation 111 locations. For example, a user or operator that does not have personal access to an automated cell engineering system facility, which may include one or more automated cell engineering system installations 111, may wish to order several cell production orders based on recently collected cell samples. The user or operator may access the capacity manager 2060 to determine which automated cell engineering system installation 111 locations have the capacity (i.e., empty automated cell engineering systems 600) and the capability (i.e., ability to conduct certain cell culture growth protocols) to fulfill the production order.

The ccps user interface manager 2062 is a software protocol in operation on central control process system 1002. The ccps user interface manager 2062 is configured to provide a user interface to allow user interaction with the central control process system 1002. The ccps user interface manager 2062 is configured to receive input from any user input source, including but not limited to touchscreens, keyboards, mice, controllers, joysticks, voice control. The ccps user interface manager 2062 is configured to provide a user interface, such as a text based user interface, a graphical user interface, or any other suitable user interface. The ccps user interface manager 2062 is configured to use the ccps network manager 2052 to provide such user interface services through one or more clients 104. The ccps user interface manager 2062 may be configured to provide different user interface services depending on a type of client device. For example, a laptop or desktop computer may be provided with a user interface including a full suite of interface options, while a smartphone or tablet may be provided with a user interface limited to status updates.

The ccps user interface manager 2062 is further configured to provide user authentication services and access management services. The ccps user interface manager 2062 is configured to manage user authentication and access management at any of the automated process control systems 102 and/or any automated cell engineering system 600 or automated cell engineering system installation 111 associated with the central control process system 1002 connected network according to any of the functionality described above with respect to the apcs user interface manager 255. The ccps user interface manager 2062 is thus configured to control access and update, alter, or otherwise adjust user access credentials to any automated cell engineering system 600 within the central control process system 1002 connected network. As used herein, the “connected network” refers to the constellation of central control process systems 1002, automated process control systems 102, automated cell engineering systems 600, and automated cell engineering system installations 111 connected via network connections. The ccps user interface manager 2062 is further configured to control access, provide user authentication services, and manage user access records to the central control process system 1002 itself according to any of the functionality described herein with respect to the apcs user interface manager 255.

The ccps data storage manager 2064 is a software protocol in operation on central control process system 1002. The ccps data storage manager 2064 is configured to access one or more automated cell engineering system 600, automated cell engineering system installation 111, and/or automated process control system 102 to receive and/or retrieve automated cell engineering system data. Automated cell engineering system data may include, for example, production data, which may be obtained in near real time, archived data, and/or data extracts, as well as process information, process parameter information, and any other information collected from one or more automated cell engineering system 600. The ccps data storage manager 2064 is further configured to access one or more data retention systems 190 and the central data retention system 1090 to store and/or receive automated cell engineering system data.

The ccps data storage manager 2064 may provide data to a user via the ccps user interface manager 2062. In embodiments, the ccps data storage manager 2064 is further configured to provide access tools to the user to manage, access, and analyze automated cell engineering system data. For example, the ccps data storage manager 2064 may be configured to generate reports, collate automated cell engineering system data, cross-reference automated cell engineering system data, populate databases with automated cell engineering system data, etc.

In embodiments, the ccps data storage manager 2064 may be configured to store and manage data records in compliance with Federal Regulations such as 21 C.F.R. part 11. For example, ccps data storage manager 2064 may implement user access controls, data validation checks, archival backups, data reproductions, data auditing, and other processes in compliance with Federal Regulations. Furthermore, the ccps data storage manager 2064 may be configured to audit, review, and otherwise check one or more automated process control systems 102 to determine compliance with appropriate Federal Regulations.

FIG. 12 is a flow chart showing a process 1200 of controlling a plurality of automated process control systems via a central control process system. The process 1200 is performed on a computer system having one or more physical processors programmed with computer program instructions that, when executed by the one or more physical processors, cause the computer system to perform the method. The one or more physical processors are referred to below as simply the processor. In embodiments, the process 1200 is carried out via the central control process system 1002 as described herein. The central control process system 1002 represents an example of a hardware and software combination configured to carry out process 1200, but implementations of the process 1200 are not limited to the hardware and software combination of the central control process system 1002. Additional details regarding each of the operations of the method may be understood according to the description the central control process system 1002, as described above.

In an operation 1202, process 1200 includes establishing a network connection with an automated cell engineering system. A network connection between a central control process system as described herein and a plurality of automated process control systems as described herein may be established via any suitable network transmission protocol or protocol suite, including, e.g., http, TCP/IP, LAN, WAN, WiFi, etc.

In an operation 1204, process 1200 includes accessing control information history of at least one automated process control system from the plurality of connected automated process control systems. As described above, control information history includes a log of control information and associated users. Operation 1204 may further include accessing any and all automated cell engineering system data stored in data retention systems 190 associated with the automated process control system.

In an operation 1206, process 1200 includes providing at least one of a cell culture growth protocol update and a cell engineering software update to the at least one automated process control system. In embodiments, the cell culture growth protocol update and/or the cell engineering software update may be provided to any number of automated process control systems 102 to which the central control process system 1002 is connected, including all automated process control systems 102.

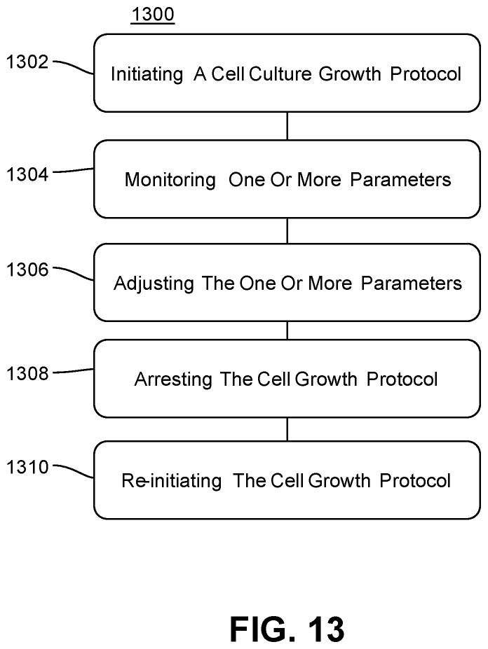

FIG. 13 is a flow chart showing a process 1300 of controlling production of a cell culture. Aspects of the process 1300 may be performed by a computer system having one or more physical processors programmed with computer program instructions that, when executed by the one or more physical processors, cause the computer system to perform the method. Further aspects of the process 1300 may be performed by an automated cell engineering system. The one or more physical processors are referred to below as simply the processor. In embodiments, the process 1300 is carried out via the automated process control system 102 or central control process system 1002 as described herein in conjunction with an automated cell engineering system 600. In embodiments, the process 1300 is carried out during cell culture growth processes that require the arrest and re-initiation of a cell culture growth protocol, as described below. Additional details regarding each of the operations of the method may be understood according to the descriptions of the automated process control system 102 and the central control process system 1002, as described above.

In an operation 1302, process 1300 includes initiating a cell culture growth protocol within the automated cell engineering system. The cell culture growth protocol may be initiated at an automated cell engineering system directly or through a control system such as an automated process control system. Cell culture growth protocol initiation may be performed according to methods and techniques discussed herein.

In an operation 1304, process 1300 includes monitoring process information of the cell culture growth protocol. As described herein, process information may include one or more cell growth parameters, including at least one of temperature information, pH information, glucose concentration information, oxygen concentration information, component or patient identification information, optical density information, and any other process information collected. In embodiments, production information may also be monitored. Monitoring of this information may collectively provide information regarding the progress of the cell culture growth protocol. The process information and/or the production information may be monitored, for example, via a control system such as an automated process control system.

In an operation 1306, process 1300 includes adjusting one or more process parameters of the cell culture growth protocol based on the monitoring. The process parameters may be adjusted to cause changes in the values measured by the process information. Process parameter adjustment may be performed by an automated process control system as discussed herein.

In an operation 1308, process 1300 includes arresting the cell culture growth protocol and recording a stage within the cell culture growth protocol at which the arresting occurred. Arresting the cell culture growth protocol may be performed by the automated process control system initiating cell growth arresting procedures within the automated cell engineering system. Such growth arresting suitable includes stopping introduction of new cell growth media, stopping introduction of cellular nutrients, or can include adjusting gas concentrations and/or temperatures to halt cell growth. Operation 1308 further includes recording the stage within the cell culture growth protocol at which the growth was arrested. By recording the stage within the cell culture growth protocol, the system may facilitate the re-initiation of the cell culture growth protocol. In some embodiments, the system may permit the cell culture growth protocol to continue to a point within the protocol that facilitates arresting of the cell culture growth protocol.

Arresting a cell culture growth protocol may be performed with various goals. For example, it may be desired to delay full cell growth to better coincide with a patient treatment plan—particularly where the treatment plan may have changed. In another example, monitoring of the process information and production information may have revealed a deficiency or anomaly in the performance of the automated cell engineering system. Arresting the cell culture growth protocol may therefore permit transferring the cell culture from one automated cell engineering system to another automated cell engineering system prior to re-initiation. In another example, cell growth may be arrested to permit trouble-shooting of potential problems within an automated cell engineering system.

In an operation 1310, process 1300 includes re-initiating the cell culture growth at the recorded stage within the cell culture growth protocol. Operation 1310 permits the automated cell engineering system, whether the original automated cell engineering system or a new automated cell engineering system, to resume the cell culture growth protocol at a same point in the process as the growth was arrested. Re-initiating the cell culture growth protocol can include providing new cell growth media, modifying gas concentrations or temperatures to re-initiate cell culture growth.

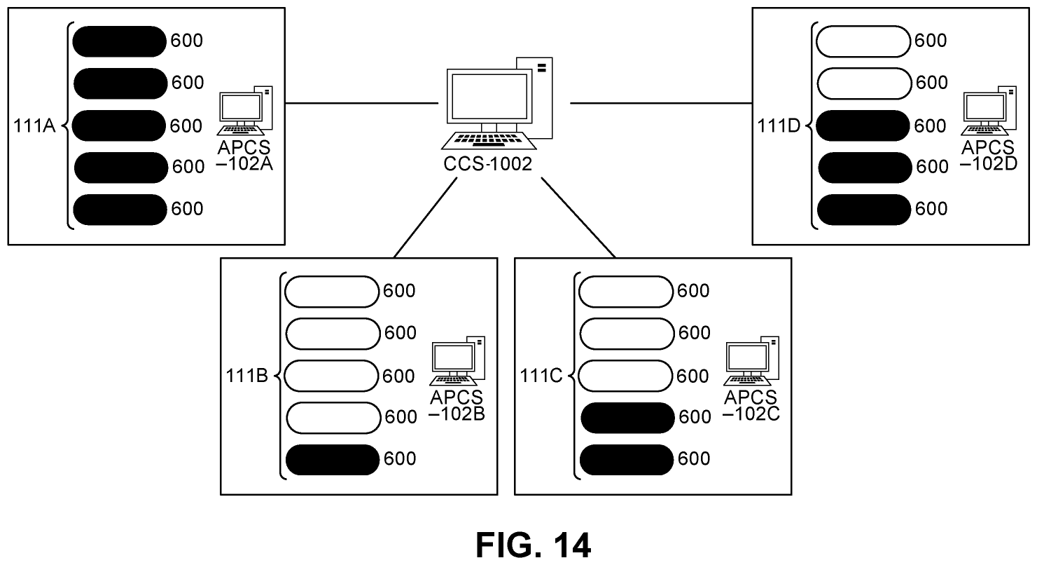

FIG. 14 illustrates a capacity utilization service according to embodiments hereof. Automated cell engineering systems 600 as controlled by automated process control system 102 and/or central control process systems 1002, as described herein, separates the geographical location of automated cell engineering systems 600 from the controlling entity and from the patient location. A network of automatic cell engineering system centers or installations 111 having different levels of capacity may be spread throughout a city or state or country. A hospital or treatment center wishing to utilize the cell engineering system technology may access the capacity utilization system to determine which facilities have excess capacity and thereby arrange for the use of the excess physical capacity. The treatment center taking advantage of the excess physical capacity may, through the use of the central control process system 1002, retain process control or monitoring without physical collocation.

The capacity utilization service operates on the central control process system 1002, and particularly, via the capacity manager 2060. As shown in FIG. 14, the central control process system 1002 may connect to multiple automated process control systems 102A, 102B, 102C, 102D. Each automated process control system 102 may be connected to multiple automated cell engineering systems 600 (e.g., an automated cell engineering system installation 111). The automated process control system 102 stores utilization information indicative of the current state of utilization of each automated cell engineering system 600 to which it is connected. The utilization information includes information about which automated cell engineering systems 600 are occupied, about the cell culture growth protocols that are currently being run in the occupied automated cell engineering system 600, and about programmed production orders that may occupy an automated cell engineering system 600 in the future but that have not yet begun processing. The capacity manager 2060, as described above, receives the utilization information from each automated process control system 102 to determine system-wide available capacity. FIG. 14 shows varying levels of utilization in the automated cell engineering systems 600, ranging from full (automated cell engineering system 600A) to partially utilized (automated cell engineering systems 600B, 600C, and 600D).

A user may access the capacity manager 2060, e.g., through a client 1004 configured for interface with the central control process system 1002 or through a client 104 configured for interface with an automated process control system 102. The user may provide information to the capacity manager 2060 about a desired production order and the capacity manager 2060 may determine which automated cell engineering system facility has excess capacity among the one or more automated cell engineering system installations located therein. The user may then arrange to deliver one or more biological samples to the selected automated cell engineering system facility for production of a cell culture. The user may then use either the central control process system 1002 or the automated process control system 102 to which they have access to monitor the cell culture growth. Through the central control process system 1002 or the automated process control system 102, the user may access the local data retention system 190 associated with the automated cell engineering system facility at which the cell culture is being produced.

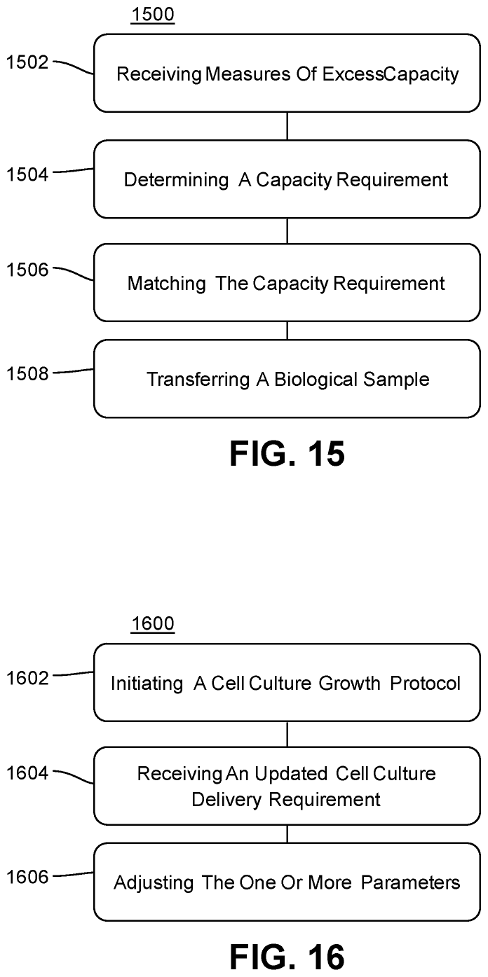

FIG. 15 is a flow chart showing a process 1500 for utilizing excess capacity within a network of automated cell engineering system configured for automated production of cell cultures. Aspects of the process 1500 may be performed by a computer system having one or more physical processors programmed with computer program instructions that, when executed by the one or more physical processors, cause the computer system to perform the method. Further aspects of the process 1500 may be performed by an automated cell engineering system, such as automated cell engineering system 600 as described herein. The one or more physical processors are referred to below as simply the processor. In embodiments, the process 1500 is carried out via the automated process control system 102 or central control process system 1002 as described herein in conjunction with one or more automated cell engineering system 600. Additional details regarding each of the operations of the method may be understood according to the descriptions of the automated process control system 102 and the central control process system 1002, as described above. Each of the process steps as described below may be performed locally via an automated process control system 102 and/or centrally by a central control process system 1002. Any combination of the steps may be performed by the automated process control system 102, the automated cell engineering system, and/or the central control process system 1002.

In an operation 1502, process 1500 includes receiving, from a plurality of automated process control stations within the network, measures of excess capacity of the automated cell engineering systems. Capacity refers to available space in an automated cell engineering system or automated cell engineering system installation within a facility that may be used to produce a cell culture. In embodiments, measures of capability are also received. Capability refers to the ability at a particular facility associated with an automated cell engineering system to carry out a given cell culture growth protocol. Capability at a facility may be limited by available supplies and available cell culture growth protocols. The measures of excess capacity may be derived from a combination of current capacity utilization and predicted capacity utilization, as described above. Predicted capacity utilization may be determined according to currently running cell culture growth protocols and future production orders. The measures of excess capacity may be computed by a local automated process control system and communicated to the central control process system. In further embodiments, the measures of excess capacity may be computed by the central control process system based on automated cell engineering system data received from the automated process control system. The measures of excess capacity may be provided to any appropriate users, including physicians, clinicians, patients, hospital administrators, etc. The measure of excess capacity can be provided to such users by various methods, including for example, via mobile device (e.g., smart phone or tablet), or to a centralized system or clinical control site (e.g., a hospital site or clinical hub), or to a database which can then be accessed by one or more of the users described herein.

In an operation 1504, process 1500 includes determining a capacity requirement according to patient requirements for a cell culture. Capacity requirements may be determined according to production orders, for example. In embodiments, capability requirements are also determined. Based on the patient cell culture requirement, the system (e.g., the automated process control system or central control process system) determines one or both of capacity and capability needs to produce the required cell cultures.

In an operation 1506, process 1500 includes matching the capacity requirement to a selected automated cell engineering system according to the measures of excess capacity. In embodiments, capability requirements are also matched. Matching the requirements includes determining which automated cell engineering system facilities have available capacity and capability that matches those required to produce the patient cell culture. Matching the requirements may further include selecting one or more automated cell engineering system at one or more facilities to conduct the required cell culture production. These matching requirements can also be provided to users (e.g., hospitals, doctors, clinics, etc.) by various methods, including for example, via mobile device (e.g., smart phone or tablet), or to a centralized system or clinical control site (e.g., a hospital site or clinical hub), or to a database which can then be accessed by one or more of the users described herein.

In an operation 1508, process 1500 includes transferring a biological sample to the selected cell engineering system for production of a cell culture. Biological sample transfer may include transfer to a selected facility that meets the determined capability and capacity requirements. One or more biological samples may be transferred to the cell engineering system and a cell culture growth protocol may be initiated to produce the required patient cell culture. In embodiments, a user that requested the transfer of biological samples is provided with authorized access to the automated process control system associated with the automated cell engineering system to which the biological samples were transferred. The user may be granted access to only those records and functions that pertain to the transferred samples. Accordingly, the user may monitor and as required, alter the process parameters of the automated cell engineering system within which the requested cell culture is being produced.

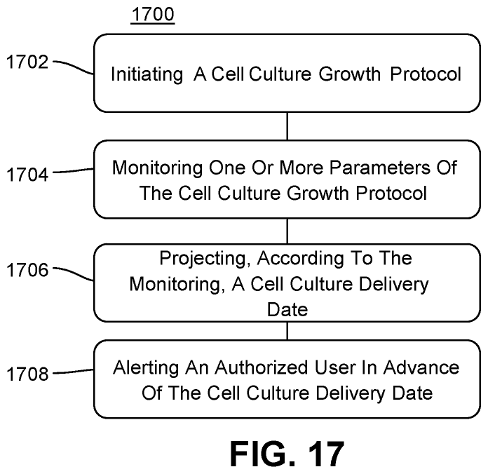

As discussed above, automated cell engineering systems consistent with embodiments described herein permit in-situ alterations to cell culture growth protocols through a combination of the automated process control system 102, central control process system 1002, client 104, and client 1004. An authorized user may update, adjust, or otherwise alter a cell culture growth protocol or automated cell engineering system process parameters during cell production. Further, systems provided herein may provide feedback providing information about cell production, i.e., production information. Thus, the systems described herein provide an increased level of interactivity between a user (such as a doctor or other treatment specialist) and the cell growth process. Changing patient requirements may therefore be used to alter and adjust cell growth, while cell growth information may be used to alter and adjust patient treatment plans, each of these alterations or adjustments being potentially subject to review by quality assurance operators. FIGS. 16 and 17 illustrate example processes of such interactions.

FIG. 16 is a flow chart showing a process 1600 for automated production of a cell growth culture performed in an automated cell engineering system. In the process 1600, cell growth parameters are altered in view of patient needs and/or doctor recommendations. Such alterations may be performed in view of a patient's changing condition and/or prognosis. For example, where a patient has unexpectedly sickened, it may be necessary to provide treatment earlier than originally anticipated. Accordingly, it may be necessary to alter a cell culture growth protocol to encourage faster cellular growth.

Aspects of the process 1600 may be performed by a computer system having one or more physical processors programmed with computer program instructions that, when executed by the one or more physical processors, cause the computer system to perform the method. Further aspects of the process 1600 may be performed by an automated cell engineering system, such as the automated cell engineering system 600 as described herein. The one or more physical processors are referred to below as simply the processor. In embodiments, the process 1600 is carried out via the automated process control system 102 or central control process system 1002 as described herein in conjunction with one or more automated cell engineering system 600. Additional details regarding each of the operations of the method may be understood according to the descriptions of the automated process control system 102 and the central control process system 1002, as described above. Each of the process steps as described below may be performed locally via an automated process control system 102, the automated cell engineering system, and/or centrally by a central control process system 1002. Any combination of the steps may be performed by the automated process control system 102 and/or the central control process system 1002.

In an operation 1602, process 1600 includes initiating a cell culture growth protocol within the automated cell engineering system. The cell culture growth protocol may be initiated at an automated cell engineering system directly or through a control system such as an automated process control system and/or through a central control process system. Cell culture growth protocol initiation may be performed according to methods and techniques discussed herein.

In an operation 1604, process 1600 includes receiving, from an authorized user, an updated cell culture delivery requirement. The updated cell culture delivery requirement may include updates to a date of delivery, updates to the number of required cells, and/or updates to particular cellular characteristics, including transformation characteristics of the cells (e.g., what gene or genes the cells may carry), antibody expression characteristics, etc.

In an operation 1606, process 1600 includes adjusting one or more parameters of the cell culture growth protocol based on the updated cell culture delivery requirement. Parameters of the cell culture growth protocol, i.e. process parameters, may be adjusted based on the updated cell culture delivery requirement so as to better meet the requirement. For example, if more cells or an earlier completion date are required, process parameters may be adjusted to accelerate the growth of cells, such as increasing feeding conditions or cell culture characteristics, temperature, gas exchange, etc.

FIG. 17 is a flow chart showing a process 1700 for automated production of a cell growth culture performed in an automated cell engineering system. In the process 1700, patient interactions, treatments, etc., may be scheduled or otherwise driven by updates and reports from the automated cell engineering system. As cell growth continues, either on schedule or not, reports on the timing of cell readiness from the cell engineering systems may be used by doctors or treatment specialists to tailor patient treatment to ready patients for treatment when cell growth is complete.

Aspects of the process 1700 may be performed by a computer system having one or more physical processors programmed with computer program instructions that, when executed by the one or more physical processors, cause the computer system to perform the method. Further aspects of the process 1700 may be performed by an automated cell engineering system, such as the automated cell engineering system 600 described herein. The one or more physical processors are referred to below as simply the processor. In embodiments, the process 1700 is carried out via the automated process control system 102, the automated cell engineering system, or central control process system 1002 as described herein in conjunction with one or more automated cell engineering system 600. Additional details regarding each of the operations of the method may be understood according to the descriptions of the automated process control system 102 and the central control process system 1002, as described above. Each of the process steps as described below may be performed locally via an automated process control system 102, an automated cell engineering system, and/or centrally by a central control process system 1002. Any combination of the steps may be performed by the automated process control system 102 and/or the central control process system 1002.

In an operation 1722, process 1700 includes initiating a cell culture growth protocol within the automated cell engineering system. The cell culture growth protocol may be initiated at an automated cell engineering system directly or through a control system such as an automated process control system and/or through a central control process system. Cell culture growth protocol initiation may be performed according to methods and techniques discussed herein.

In an operation 1724, process 1700 includes monitoring process information and/or production information of the cell culture growth. As described herein, process information may include at least one of temperature information, pH information, glucose concentration information, oxygen concentration information, optical density information, component or patient identification information, and any other process information collected. In embodiments, production information may also be monitored. Monitoring of this information may collectively provide information regarding the progress of the cell culture growth protocol. The process information and/or the production information may be monitored, for example, via a control system such as an automated process control system.

In an operation 1726, process 1700 includes projecting, according to the monitoring, a cell culture delivery date. A cell culture delivery date refers to a date and time at which the production of a cell culture has progressed to a point at which it is suitable for use as desired, including for administration to a patient. An automated process control system or central control process system may project, based on one or more of the process information, production information, and cell culture growth protocol, when production of a required number of cells is complete for cell culture delivery. An initial prediction of a cell culture delivery date may be based on the cell culture growth protocol. This prediction may be updated based on process information, for example, if process variables differ from cell culture growth protocol specifications in a way that will speed up or slow down cell culture growth. This prediction may also be updated based on production information, for example, if cell culture growth is proceeding faster or more slowly than initially anticipated.

In an operation 1728, process 1700 includes notifying an authorized user in advance of the cell culture delivery date. Notifications may be provided via e-mail, text message, and/or messaging within the computing environment provided by the automated process control system and/or central control process system. Notifications may be provided one or more days in advance of an anticipated cell culture delivery date. Physicians may use this information to schedule and organize patient treatment schedules. Authorized users may include, for example, physicians, patients, clinicians, administrative staff, and any other personnel involved in cell culture production and patient treatment. Notifications can also be provided to a centralized hospital or clinical hub that may be overseeing the process.

In some aspects and as described, the automated cell engineering system 600 may include a user interface 1130 that can include a component identification sensor such as a bar code reader, QR code reader, radio frequency ID interrogator, or other component identification sensor. In some aspects, a cassette 602 can include a first identification component, such as a bar code, and the user interface 1130 can include a reader that is configured to read and identify the first identification component. In some aspects, the automated cell engineering system 600 user interface can initiate a handshake interrogation between the cassette 602 and the user interface 1130 whereby the automated cell engineering system 600 is able to verify that the cassette utilized is an authorized component, is the proper cassette for the protocol selected to be run on the automated cell engineering system 600, or otherwise is correctly paired to the automated cell engineering system 600. Handshake interactions between automated cell engineering system 600 and the cassette 602 may be monitored, reviewed, recorded, and otherwise checked by the automated process control system 102 and/or the central control process system 1002.

In some aspects, this procedure can allow for proper equipment authentication as may be required by applicable law, such as 21 C.F.R. part 11. Further, and for example in facilities with multiple automated cell engineering systems 600 operating simultaneously, the automated cell engineering system 600 can be configured to store the component and protocol identification either locally on the automated cell engineering system 600 or remotely in a database that is accessed via the above described information pathways.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

Further specific embodiments include:

Embodiment 1 is a method of controlling an automated cell engineering system configured to produce a cell culture, the method comprising: establishing, by an automated process control system, a network connection with the automated cell engineering system; receiving, via the network connection, process information from the automated cell engineering system, the process information including one or more of temperature information, pH information, glucose concentration information, oxygen concentration information, component or patient identification information, and optical density information; providing a control signal to cause the automated cell engineering system to adjust one or more process parameters of the automated cell engineering based on the received process information.

Embodiment 2 is the method of embodiment 1, further comprising providing a plurality of additional control signals to a plurality of additional cell engineering systems via a plurality of additional network connections.

Embodiment 3 is the method of embodiments 1 or 2, wherein the cell culture is a genetically modified cell culture.

Embodiment 4 is the method of embodiments 1 to 3, wherein the cell culture is a genetically modified immune cell culture.

Embodiment 5 is the method of embodiments 1 to 4, wherein providing the control signal is performed without user intervention.

Embodiment 6 is the method of embodiments 1 to 5, wherein providing the control signal is performed based on user authorization.

Embodiment 7 is the method of embodiments 1 to 6, further including receiving production information including cell production information recorded over time, the method further comprising storing, in a local database, the production information.

Embodiment 8 is the method of embodiments 1 to 7, further comprising monitoring, via the automated process control system, a handshake interrogation procedure performed by the automated cell engineering system responsive to the introduction of a cassette.

Embodiment 9 is the method of embodiments 1 to 8, wherein the control signal is generated at the automated cell engineering system via operator interaction at the automated cell engineering system.

Embodiment 10 is a method of controlling a plurality of automated process control systems via a central control system, the method comprising: establishing network connections with a plurality of computer systems corresponding to a plurality of automated process control systems, each configured to control a plurality of automated cell engineering systems configured for production of cell cultures; accessing, by the central control system, control information history of a first computer system from the plurality of computer systems; and providing to the first computer system at least one of a cell culture growth protocol update and a cell engineering software update.

Embodiment 11 is the method of embodiment 10, further comprising providing the cell engineering software update to the plurality of computer systems.

Embodiment 12 is the method of embodiment 10 or 11, further comprising analyzing the control information history; and modifying local user access to the first computer system based on the analysis of the control information history.

Embodiment 13 is the method of embodiments 10 to 12, further comprising analyzing the control information history to determine local user compliance with best practices or ethical guidelines.

Embodiment 14 is a method for automated production of a cell culture performed by an automated cell engineering system, the method comprising: initiating a cell culture growth protocol within the automated cell engineering system; monitoring process information of the cell culture growth protocol; adjusting one or more parameters of the cell culture growth protocol based on the monitoring; arresting the cell culture growth protocol and recording a stage within the protocol at which the arresting occurred; and re-initiating the cell culture growth protocol at the stage within the cell culture growth protocol.

Embodiment 15 is the method of embodiment 13, further comprising transferring a cell culture from a first cell engineering system to a second cell engineering system after the arresting and prior to the re-initiating.

Embodiment 16 is a method for utilizing excess capacity within a network of automated cell engineering systems configured for automated production of cell cultures, the method comprising: receiving, from a plurality of automated process control systems within the network, measures of excess capacity of the automated cell engineering systems; determining a capacity requirement according to patient requirements for a cell culture; matching the capacity requirement to a selected automated cell engineering system according to the measures of excess capacity; and transferring a biological sample to the selected cell engineering system for production of a cell culture.

Embodiment 17 is a method for automated production of a cell culture performed by an automated cell engineering system, the method comprising: initiating a cell culture growth protocol within the automated cell engineering system; receiving, from an authorized user, an updated cell culture delivery requirement; and adjusting one or more parameters of the cell culture growth protocol based on the updated cell culture delivery requirement.

Embodiment 18 is a method for automated production of a cell culture performed by an automated cell engineering system, the method comprising:

initiating a cell culture growth protocol within the automated cell engineering system; monitoring one or more parameters of the cell culture growth protocol; projecting, according to the monitoring, a cell culture delivery date; and alerting an authorized user in advance of the cell culture delivery date.

All publications, patents and patent applications mentioned in this specification are herein incorporated by reference to the same extent as if each individual publication, patent, or patent application was specifically and individually indicated to be incorporated by reference.

1. A method of controlling an automated cell engineering system configured to produce a cell culture, the method comprising:
establishing, by an automated process control system, a network connection with the automated cell engineering system;
receiving, via the network connection, process information from the automated cell engineering system, the process information including one or more of temperature information, pH information, glucose concentration information, oxygen concentration information, component identification information, and optical density information; and
providing a control signal to cause the automated cell engineering system to adjust one or more process parameters of the automated cell engineering based on the process information.

2. The method of claim 1, further comprising providing a plurality of additional control signals to a plurality of additional cell engineering systems via a plurality of additional network connections.

3. The method of claim 1, wherein the cell culture is a genetically modified cell culture.

4. The method of claim 1, wherein the cell culture is a genetically modified immune cell culture.

5. The method of claim 1, wherein providing the control signal is performed without user intervention.

6. The method of claim 1, wherein providing the control signal is performed based on user authorization.

7. The method of claim 1, further including receiving production information including cell production information recorded over time, the method further comprising storing, in a database, the production information.

8. The method of claim 1, further comprising monitoring, via the automated process control system, a handshake interrogation procedure performed by the automated cell engineering system responsive to introduction of a cassette.

9. The method of claim 1, wherein the control signal is generated at the automated cell engineering system via operator interaction at the automated cell engineering system.

10. A method of controlling a plurality of automated process control systems via a central control system, the method comprising:
establishing network connections with a plurality of computer systems corresponding to a plurality of automated process control systems, each configured to control a plurality of automated cell engineering systems configured for production of cell cultures;
accessing, by the central control systems, control information history of a first computer system from the plurality of computer systems; and
providing to the first computer system at least one of a cell culture growth protocol update and a cell engineering software update.

11. The method of claim 10, further comprising providing the cell engineering software update to the plurality of computer systems.

12. The method of claim 10, further comprising analyzing the control information history; and modifying local user access to the first computer system based on the analyzing of the control information history.

13. The method of claim 10, further comprising analyzing the control information history to determine local user compliance with best practices or ethical guidelines.

14. A method for automated production of a cell culture performed by an automated cell engineering system, the method comprising:
initiating a cell culture growth protocol within the automated cell engineering system;
monitoring process information of the cell culture growth protocol;
adjusting one or more parameters of the cell culture growth protocol based on the monitoring;
arresting the cell culture growth protocol and recording a stage within the protocol at which the arresting occurred; and
re-initiating the cell culture growth protocol at the stage within the cell culture growth protocol.

15. The method of claim 14, further comprising transferring a cell culture from a first cell engineering system to a second cell engineering system after the arresting and prior to the re-initiating.

16. A method for utilizing excess capacity within a network of automated cell engineering systems configured for automated production of cell cultures, the method comprising:
receiving, from a plurality of automated process control systems within the network, measures of excess capacity of the automated cell engineering systems;
determining a capacity requirement according to patient requirements for a cell culture;
matching the capacity requirement to a selected automated cell engineering system according to the measures of excess capacity; and
transferring a biological sample to the selected cell engineering system for production of a cell culture.

17. A method for automated production of a cell culture performed by an automated cell engineering system, the method comprising:
initiating a cell culture growth protocol within the automated cell engineering system;
receiving, from an authorized user, an updated cell culture delivery requirement; and
adjusting one or more parameters of the cell culture growth protocol based on the updated cell culture delivery requirement.

18. A method for automated production of a cell culture performed by an automated cell engineering system, the method comprising:
initiating a cell culture growth protocol within the automated cell engineering system;
monitoring one or more parameters of the cell culture growth protocol;
projecting, according to the monitoring, a cell culture delivery date; and
alerting an authorized user in advance of the cell culture delivery date.
